(19) United States
(12) Patent Application Publication
(10) Pub. No.: US 2023/0072934 A1
(43) Pub. Date: Mar. 9, 2023

(54) SIGNAL RESTORATION SYSTEM, SIGNAL RESTORATION METHOD, COMPUTER PROGRAM, AND SIGNAL GENERATION SYSTEM USING AI

(71) Applicant: Data Solutions, Inc., Tokyo (JP)

(72) Inventors: Tomoaki OTSUKI, Yokohama-shi, Kanagawa (JP); Kohei YAMAMOTO, Yokohama-shi, Kanagawa (JP); Shuzo ISHIZAKA, Yokohama-shi, Kanagawa (JP); Ryosuke HIROMATSU, Yokohama-shi, Kanagawa (JP)

(73) Assignee: Data Solutions, Inc., Tokyo (JP)

(21) Appl. No.: 17/904,686

(22) PCT Filed: Feb. 18, 2021
(86) PCT No.: PCT/JP2021/006203

§ 371 Date: Aug. 19, 2022

(30) Foreign Application Priority Data
Feb. 21, 2020 (JP) 2020-028681

(51) Int. Cl. A61B 5/00; A61B 5/05; A61B 5/11

(52) U.S. Cl. CPC A61B 5/7267; A61B 5/05; A61B 5/725; A61B 5/7242; A61B 5/1102; A61B 5/1128; A61B 5/7278; A61B 5/7203; A61B 5/02125

(57) ABSTRACT

A signal representing heartbeat behavior is accurately restored. The present signal restoration system includes: a signal acquirer configured to acquire a first heartbeat signal representing heartbeat behavior; a first band-pass filter configured to generate a first signal by performing first band-pass filter processing on the first heartbeat signal; an integral calculator configured to calculate an integral value by integrating frequency intensity of the heartbeat represented by the first signal; a second band-pass filter configured to generate a third signal by performing second band-pass filter processing on a second signal representing the integral value with respect to time; and a restored signal generator configured to generate a restored signal representing heartbeat behavior based on first data generated by dividing the third signal at intervals of a predetermined time.

### TECHNICAL FIELD

The present invention relates to a signal restoration system, a signal restoration method, a computer program, and a signal generation system using AI.

### BACKGROUND ART

In a recently known method, a signal representing living body information such as heartbeat behavior is restored by using artificial intelligence (AI) from data obtained by measuring a subject.

For example, first, a system generates a signal by measuring the subject by using a Geophone sensor. Then, the system applies a recurrent neural network (RNN) to the generated signal. In this manner, an electric signal representing heart motion is restored, which is disclosed as a known method (for example, Non Patent Literature 1).

In another method, a measurement system calculates a pulse transit time (hereinafter referred to as a “PTT”) based on an aortic pulse wave measured by a Doppler radar. In particular, a method of obtaining systolic blood pressure (hereinafter referred to as “SBP”) by calculating a carotid-femoral PTT (hereinafter referred to as a “PTT_{cf}”), which is highly correlated with blood pressure, is known (for example, Non Patent Literature 2).

### CITATION LIST

### Non Patent Literature

Non Patent Literature 1: Poster: Deep ECG Estimation Using a Bed-attached Geophone, JaeYeon Park, Hyeon Chol, Wonjun Hwang, Rajesh Krishna Balan, and JeongGil Ko, MobiSys'19, Jun. 17 to 21, 2019, Seoul, Korea

Non Patent Literature 2: Non-contact Beat-to-beat Blood Pressure Measurement Using Continuous Wave Doppler Radar, Heng Zhao, Xu Gu, Hong Hong, Yusheng Li, Xiaohua Zhu, and Changzhi Li, 2018 IEEE/MTT-S International Microwave Symposium, 20 Aug. 2018.

### SUMMARY OF INVENTION

### Technical Problem

The present invention is made in view of the above-described situation and is intended to accurately restore a signal representing heartbeat behavior (also referred to as “heartbeat” or “heart behavior”; hereinafter referred to as “heartbeat behavior”).

### Solution to Problem

The present signal restoration system includes:

a signal acquirer configured to acquire a first heartbeat signal representing heartbeat behavior;

a first band-pass filter configured to generate a first signal by performing first band-pass filter processing on the first heartbeat signal;

an integral calculator configured to calculate an integral value by integrating frequency intensity of the heartbeat represented by the first signal;

a second band-pass filter configured to generate a third signal by performing second band-pass filter processing on a second signal representing the integral value with respect to time; and

a restored signal generator configured to generate a restored signal representing heartbeat behavior based on first data generated by dividing the third signal at intervals of a predetermined time.

### Advantageous Effect of Invention

According to the disclosed technology, a signal representing heartbeat behavior can be accurately restored.

### BRIEF DESCRIPTION OF DRAWINGS

FIG. 1 is a diagram illustrating an exemplary entire configuration of a first embodiment.

FIG. 2 is a diagram illustrating an exemplary Doppler radar.

FIG. 3 is a diagram illustrating an exemplary information processing device.

FIG. 4 is a diagram illustrating exemplary entire processing of the first embodiment.

FIG. 5 is a diagram illustrating an exemplary first heartbeat signal.

FIG. 6 is a diagram illustrating an exemplary spectrogram.

FIG. 7 is a diagram illustrating an exemplary integral value.

FIG. 8 is a diagram illustrating an exemplary network structure of a learning model.

FIG. 9 is a diagram illustrating an exemplary input value.

FIG. 10 is a diagram illustrating an exemplary output value.

FIG. 11 is a diagram illustrating exemplary generation of a restored signal.

FIG. 12 is a diagram illustrating an example of a Q wave, an R wave, an S wave, and a T wave in one period of heartbeat.

FIG. 13 is a diagram illustrating exemplary application of a band-pass filter of 0.5 Hz to 2.0 Hz.

FIG. 14 is a diagram illustrating exemplary application of a band-pass filter of 0.5 Hz to 10.0 Hz.

FIG. 15 is a table listing experiment specifications of the first embodiment.

FIG. 16 is a diagram illustrating a comparative example in an experiment.

FIG. 17 is a diagram illustrating peak error averages.

FIG. 18 is a diagram illustrating a comparative example of a QRS interval, a QT interval, and a RRI.

FIG. 19 is a diagram illustrating errors in the QRS interval and the QT interval.

FIG. 20 is a diagram illustrating an exemplary functional configuration in the first embodiment.

FIG. 21 is a diagram illustrating an exemplary aortic pulse wave.

FIG. 22 is a diagram illustrating an exemplary relation between “PTT_{cf}” and blood pressure.

FIG. 23 is a diagram illustrating an exemplary aortic pulse wave signal in an ideal state.

FIG. 24 is a diagram illustrating exemplary entire processing of a second embodiment.

FIG. 25 is a diagram illustrating an exemplary noise component used to generate second learning data.

FIG. 26 is a table listing conditions under which learning data of the second embodiment is generated.

FIG. 27 is a table listing conditions under which data for execution of the second embodiment is generated.

FIG. 28 is a scatter diagram of blood pressure and “PTT_{cf}” and is a diagram illustrating approximate straight lines thereof.

FIG. 29 is a diagram illustrating a calculation result of the ratio of a waveform for which a first interval “T₁” and a second interval “ED” cannot be calculated and a calculation result of a correlation coefficient.

FIG. 30 is a diagram illustrating an experiment result of error between a blood pressure of “true value” and a blood pressure indicated by an estimation result.

FIG. 31 is a diagram illustrating the experiment result of the error between the blood pressure of “true value” and the blood pressure indicated by the estimation result.

FIG. 32 is a diagram illustrating an exemplary functional configuration in the second embodiment.

FIG. 33 is exemplary IQ data measured by the Doppler radar.

FIG. 34 is a diagram illustrating an example of a result of comparison with an ECG signal.

FIG. 35 is a diagram illustrating a first estimation result.

FIG. 36 is a diagram illustrating a second estimation result.

FIG. 37 is a diagram illustrating a third estimation result.

FIG. 38 is a diagram illustrating a fourth estimation result.

FIG. 39 is a diagram illustrating a fifth estimation result.

FIG. 40 is a diagram illustrating a sixth estimation result.

FIG. 41 is a diagram illustrating a seventh estimation result.

### DESCRIPTION OF EMBODIMENTS

Optimum and minimum forms for performing the present invention will be described below with reference to the accompanying drawings. Note that, identical reference signs in the drawings denote the same component, and duplicate description thereof is omitted. Illustrated specific examples are merely exemplary, and other components than those illustrated may be included.

### First Embodiment

For example, a signal restoration system 1 is a system having an entire configuration as described below.

### Exemplary Entire Configuration

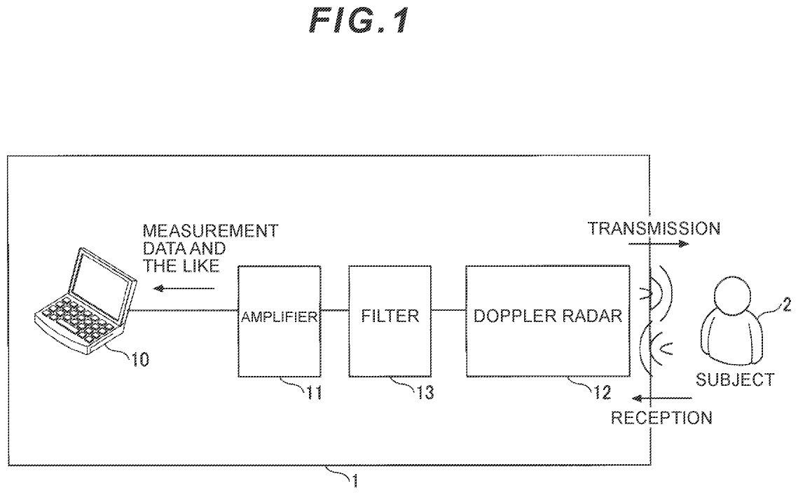

FIG. 1 is a diagram illustrating an exemplary entire configuration of a first embodiment. For example, the signal restoration system 1 has a configuration including a personal computer (PC; hereinafter referred to as a “PC 10”), a Doppler radar 12, and a filter 13. Note that, as illustrated, the signal restoration system 1 desirably has a configuration including an amplifier 11. The following description is made with the illustrated entire configuration as an example.

The PC 10 is an exemplary information processing device. The PC 10 is connected to a peripheral instrument such as the amplifier 11 through a network, a cable, or the like. Note that, the amplifier 11, the filter 13, and the like may be included in the PC 10. The amplifier 11, the filter 13, and the like may be configured not as devices but as software or as hardware and software.

The Doppler radar 12 is an exemplary measurement device.

In this example, the PC 10 is connected to the amplifier 11. The amplifier 11 is connected to the filter 13. The filter 13 is connected to the Doppler radar 12. The PC 10 acquires measurement data from the Doppler radar 12 through the amplifier 11 and the filter 13. Specifically, the measurement data is data representing heartbeat behavior. Subsequently, the PC 10 measures movement of a human body, such as a heart rate, by analyzing body motion of a subject 2, such as heartbeat, breathing, and body movement, based on the acquired measurement data.

The Doppler radar 12 acquires a signal (hereinafter referred to as a “heartbeat signal”) representing heartbeat behavior in accordance with, for example, a principle as described below.

### Exemplary Doppler Radar

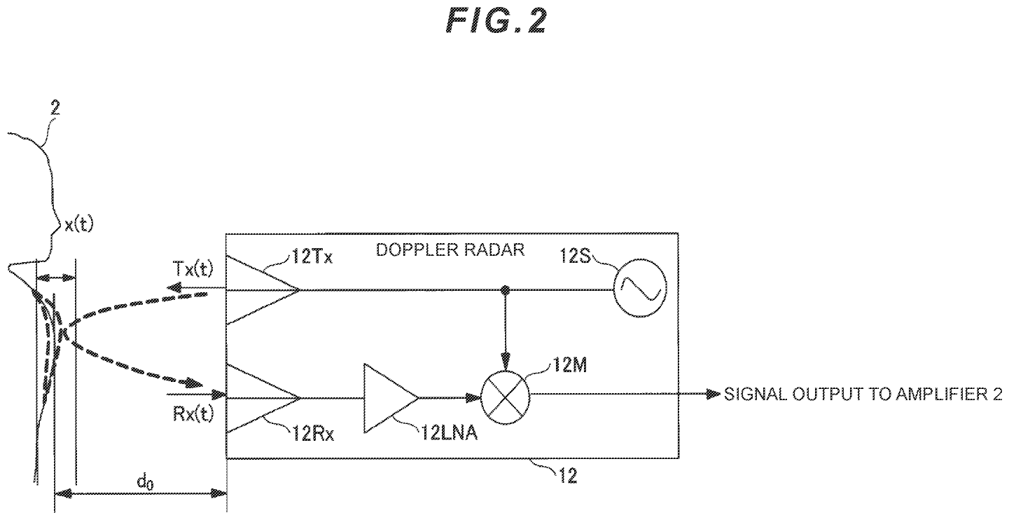

FIG. 2 is a diagram illustrating an exemplary Doppler radar. For example, the Doppler radar 12 is a device having a configuration as illustrated in FIG. 2. Specifically, the Doppler radar 12 includes a source 12S, a transmitter 12Tx, a receiver 12Rx, and a mixer 12M. The Doppler radar 12 also includes an adjuster 12LNA such as a low noise amplifier (LNA) configured to perform processing such as reduction of noise of data received by the receiver 12Rx.

The source 12S is a transmission source configured to generate a signal of transmission wave to be transmitted by the transmitter 12Tx.

The transmitter 12Tx transmits the transmission wave to the subject 2. Note that, the signal of the transmission wave can be expressed as a function Tx(t) of time t and expressed as, for example, Expression (1) below.

[Expression 1]

Tx(t)=cos(ωt) (Expression 1)

In Expression (1) above, ω_{c} represents the angular frequency of the transmission wave.

The subject 2, in other words, a reflection surface of the transmitted signal has a displacement of x(t) with respect to time t. In this example, the reflection surface is the chest wall of the subject 2. The displacement x(t) can be expressed as, for example, Expression (2) below.

[Expression 2]

x(t)=m×cos(ωt) (Expression 2)

In Expression (2) above, “m” is a constant representing the amplitude of the displacement. In Expression (2) above, “ω” is an angular velocity shifted by movement of the subject 2. Note that, any variable same as in Expression (1) above has the same meaning.

The receiver 12Rx receives reflection wave transmitted by the transmitter 12Tx and reflected by the subject 2. A signal of the reflection wave can be expressed as a function Rx(t) of time t, for example, Expression (3) below.

$$\begin{matrix}[ Expression 3 ] & \\ Rx ( t ) = cos ( {ω}_{c} t - 2 π \cdot \frac{2 ( {d}_{0} + x ( t ) )}{λ} ) & ( Expression 3 )\end{matrix}$$

In Expression (3) above, “d₀” represents the distance between the subject 2 and the Doppler radar 12. In addition, “λ” represents the wavelength of the signal. The same notations apply below.

The Doppler radar 12 generates a Doppler signal by mixing the function Tx(t) (Expression (1) above) representing a signal of transmission wave and the function R(t) (Expression (3) above) representing a signal of reception wave. Note that, the Doppler signal can be expressed as a function B(t) of time t in Expression (4) below.

$$\begin{matrix}[ Expression 4 ] & \\ B ( t ) = cos ( θ + 2 π \cdot \frac{2 x ( t )}{λ} ) & ( Expression 4 )\end{matrix}$$

When “ω_{d}” represents the angular frequency of the Doppler signal, the angular frequency ω_{d} of the Doppler signal can be expressed as Expression (5) below.

$$\begin{matrix}[ Expression 5 ] & \\ {ω}_{d} = θ + 2 π \cdot \frac{2 x ( t )}{λ} & ( Expression 5 )\end{matrix}$$

In Expressions (4) and (5) above, the phase “θ” can be expressed as Expression (6) below.

$$\begin{matrix}[ Expression 6 ] & \\ θ = 2 π \cdot \frac{2 {d}_{0}}{λ} + {θ}_{0} & ( Expression 6 )\end{matrix}$$

In Expression (6) above, “θ₀” represents the chest wall of the subject 2, in other words, phase displacement at the reflection surface.

Subsequently, the Doppler radar 12 outputs, for example, the position and speed of the subject 2 based on a result of comparison between the signal of the transmission wave thus transmitted and the signal of the reception wave thus received, in other words, a result of calculation with the above-described expressions.

For example, I data (in-phase data) and Q data (orthogonal phase data) can be generated from the reception wave. Then, a distance by which the chest wall of the subject 2 has moved can be detected based on the I data and the Q data. In addition, whether the chest wall of the subject 2 has moved forward or backward can be detected based on phases represented by the I data and the Q data. Accordingly, an indicator such as heartbeat can be detected by using frequency change in the transmission wave and the reception wave due to movement of the chest wall due to heartbeat.

### Exemplary Information Processing Device

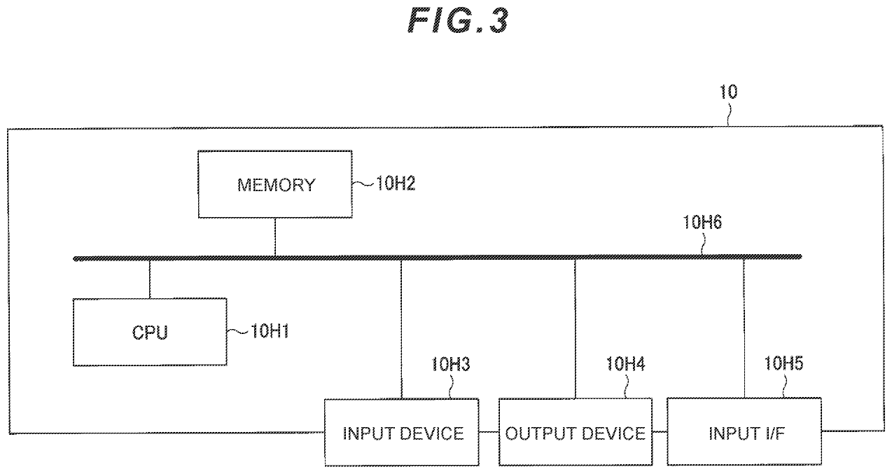

FIG. 3 is a diagram illustrating an exemplary information processing device. For example, the PC 10 includes a central processing unit (CPU; hereinafter referred to as a “CPU 10H1”), a memory 10H2, an input device 10H3, an output device 10H4, and an input interface (I/F) (hereinafter referred to as an “input I/F 10H5”). Note that, the hardware components included in the PC 10 are connected to one another through a bus 10H6, and data and the like are mutually transmitted and received among the hardware components through the bus 10H6.

The CPU 10H1 is a control device configured to control the hardware components included in the PC 10 and is an arithmetic device configured to perform calculation for achieving various kinds of processing.

The memory 10H2 is, for example, a main memory or an auxiliary memory. Specifically, the main memory is, for example, a memory. The auxiliary memory is, for example, a hard disk. The memory 10H2 stores data including intermediate data used by the PC 10, computer programs used for various kinds of processing and control, and the like.

The input device 10H3 is a device on which parameters and commands necessary for calculation are input to the PC 10 through operations by a user. Specifically, the input device 10H3 is, for example, a keyboard, a mouse, or a driver.

The output device 10H4 is a device for outputting results of various kinds of processing and calculation by the PC 10 to the user or the like. Specifically, the output device 10H4 is, for example, a display.

The input I/F 10H5 is an interface connected to an external device such as a measurement device and used to transmit and receive data and the like. The input I/F 10H5 is, for example, a connector or an antenna. In other words, the input I/F 10H5 transmits and receives data to and from the external device through a network, wireless communication, a cable, or the like.

Note that, the hardware configuration is not limited to the illustrated configuration. For example, the PC 10 may further include an arithmetic device, a memory, or the like to perform processing in a parallel, distributed, or redundant manner. The PC 10 may be an information processing system connected to another device through a network or a cable to perform calculation, control, and storage in a parallel, distributed, or redundant manner. In other words, the present invention may be achieved by an information processing system including one or more information processing devices.

As described above, the PC 10 acquires a heartbeat signal representing heartbeat behavior through the measurement device such as the Doppler radar 12. Note that, the heartbeat signal may be acquired as needed in real time or the heartbeat signal may be stored for a certain duration in a device such as the Doppler radar and thereafter collectively acquired by the PC 10. The acquisition may be performed by using a recording medium or the like.

### Exemplary Entire Processing

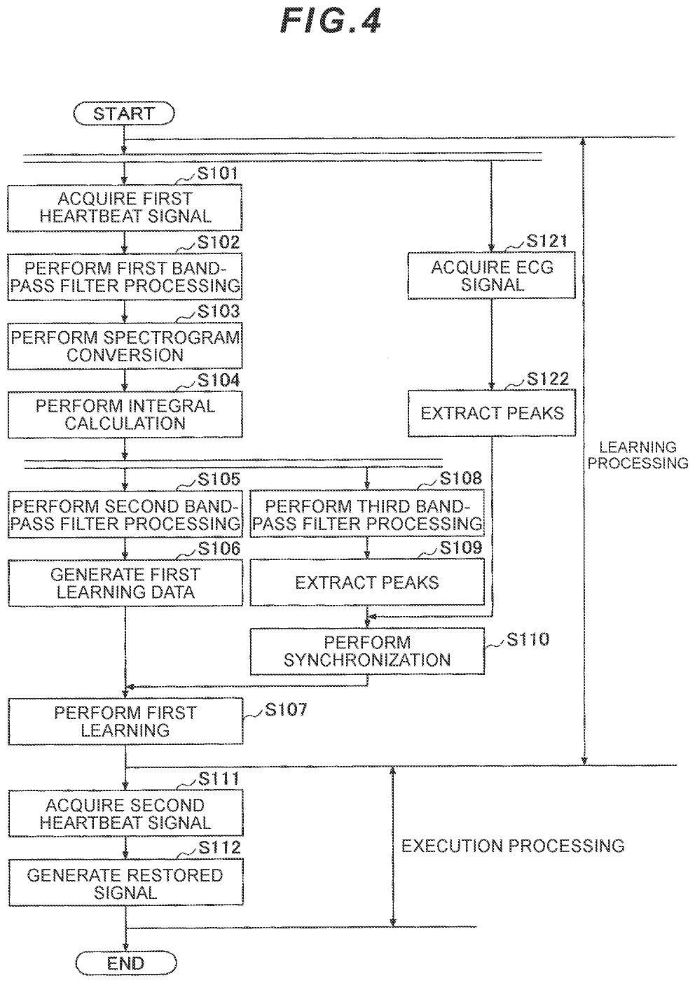

FIG. 4 is a diagram illustrating exemplary entire processing. The entire processing will be described below separately for “learning processing” and “execution processing”. Note that, the “learning processing” may be executed at any optional timing earlier than the “execution processing”. In other words, the “learning processing” and the “execution processing” do not necessarily need to be executed at continuous timings, and there may be a period before the “execution processing” is performed after the “learning processing”. The following description is made on a case in which the “execution processing” is continuously executed after the “learning processing” as an example.

### Exemplary Acquisition of First Heartbeat Signal

At step S101, the signal restoration system 1 acquires a heartbeat signal. Hereinafter, among heartbeat signals, a heartbeat signal used to generate “first learning data” as an example of first data to be described below is referred to as a “first heartbeat signal”. Accordingly, the first heartbeat signal is a signal that represents heartbeat behavior and on which learning data in machine learning is based, and is IQ data generated by the Doppler radar 12.

For example, the first heartbeat signal is a signal as described below.

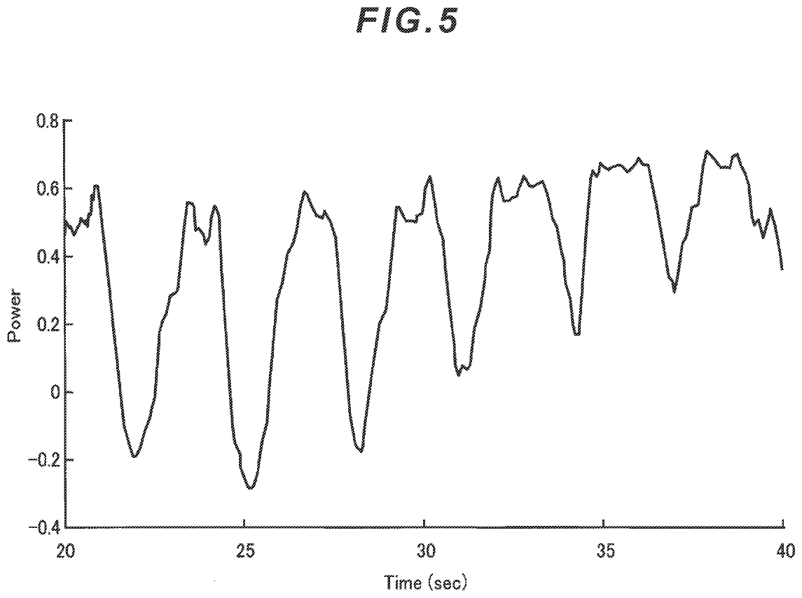

FIG. 5 is a diagram illustrating an example of the first heartbeat signal. In the drawing, the horizontal axis represents a time at which measurement is performed. The vertical axis represents electric power estimated based on a result of measurement by the Doppler radar.

### Example of First Band-Pass Filter Processing

At step S102, the signal restoration system 1 performs band-pass filter processing on the first heartbeat signal. Hereinafter, the band-pass filter processing performed on the first heartbeat signal is referred to as “first band-pass filter processing”. A signal generated by performing the first band-pass filter processing on the first heartbeat signal, in other words, a signal generated by attenuating, through the first band-pass filter processing, a signal as noise included in the first heartbeat signal is referred to as a “first signal”.

### Example of Spectrogram Conversion

At step S103, the signal restoration system 1 desirably generates a spectrogram by performing spectrogram conversion based on the first signal. For example, the spectrogram conversion is achieved by short-time Fourier transform (STFT) or the like. For example, the spectrogram is data as described below.

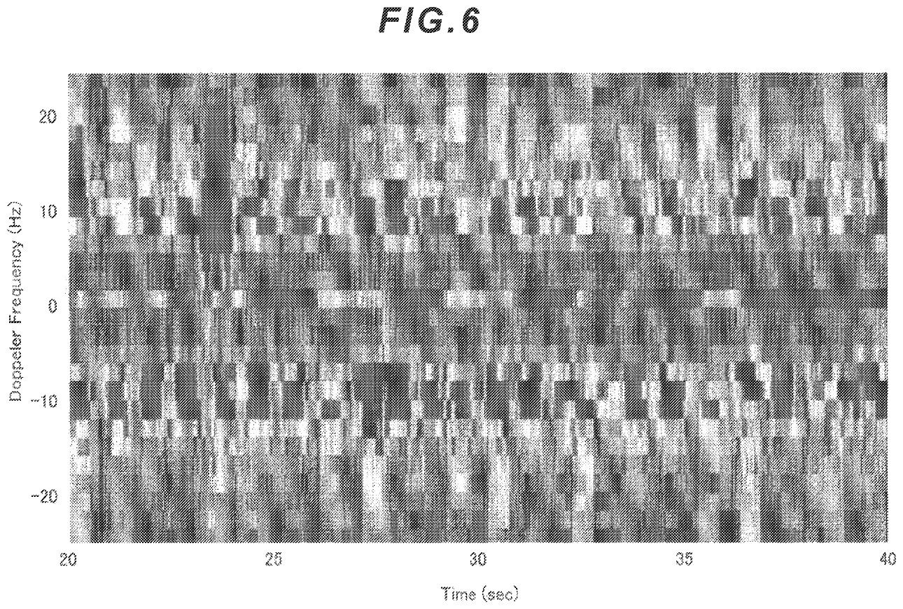

FIG. 6 is a diagram illustrating an example of the spectrogram. As illustrated, the spectrogram illustrates, for each frequency, the intensity (hereinafter referred to as “frequency intensity”) of a signal included in the first signal. In this example, the spectrogram illustrates the frequency intensity in grayscale (in this example, higher concentration represents higher intensity), and the vertical axis represents the corresponding frequency. The horizontal axis in this example represents time, and as illustrated, the spectrogram indicates the frequency intensity for each time and each frequency component. For example, the spectrogram is desirably generated in such a format.

Influence of any component other than heartbeat behavior in the heartbeat signal, in other words, noise can be reduced through such conversion to the spectrogram. Thus, data with which heartbeat behavior can be easily checked can be generated through conversion to the spectrogram.

### Example of Integral Calculation

At step S104, the signal restoration system 1 calculates an integral value of the frequency intensity based on the spectrogram. The integral calculation is performed on the intensity in the frequency domain corresponding to a heartbeat component over a range from low frequency to high frequency in the frequency domain. Specifically, the integral calculation is performed for the frequency in the range of “−30 Hz” to “−8 Hz” and in the range of “8 Hz” to “30 Hz”. The intensity corresponding to the frequency in these ranges is integrated to calculate an integral value. For example, an integral value as described below is calculated through the integral calculation.

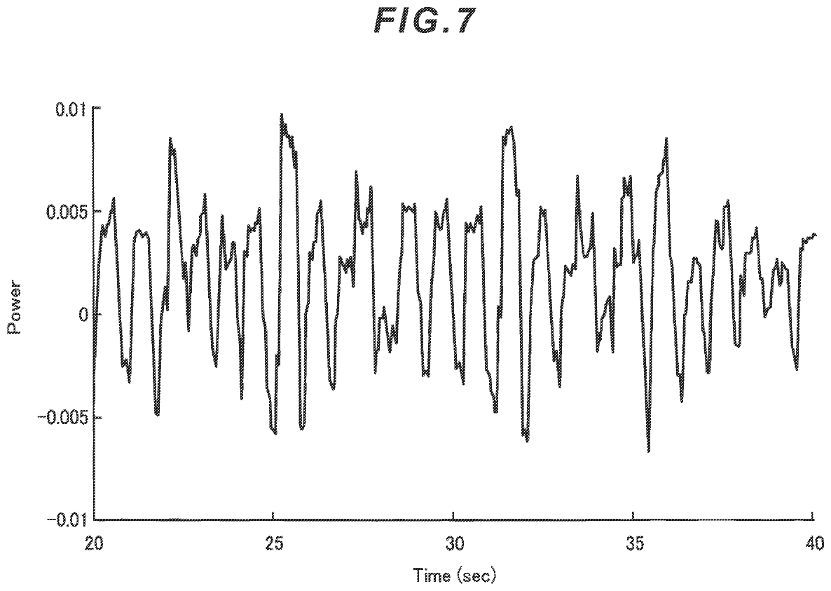

FIG. 7 is a diagram illustrating an example of the integral value. For example, when the integral calculation is performed based on the spectrogram illustrated in FIG. 6, the integral value is calculated for each time as illustrated. Hereinafter, a signal representing the integral value with respect to time as illustrated is referred to as a “second signal”. The second signal is a signal calculated at intervals of a predetermined time and representing change of the integral value with respect to time as illustrated.

Note that, no spectrogram conversion may be performed and the integral calculation may be performed on the amplitude of the first heartbeat signal as the frequency intensity.

### Example of Second Band-Pass Filter Processing

At step S105, the signal restoration system 1 performs band-pass filter processing on the second signal. Hereinafter, the band-pass filter processing performed on the second signal is referred to as “second band-pass filter processing”. Accordingly, the second band-pass filter processing is band-pass filter processing performed separately from the first band-pass filter processing and is performed on a different processing target signal at a different timing. Hereinafter, a signal generated by performing the second band-pass filter processing on the second signal, in other words, a signal generated by attenuating, through the second band-pass filter processing, a signal as noise included in the second signal is referred to as a “third signal”.

### Exemplary First Learning Data Generation

At step S106, the signal restoration system 1 generates learning data. Hereinafter, learning data to be used as an input in first learning executed later at step S107 is referred to as “first learning data”. For example, the first learning data is generated by dividing the third signal at intervals of a predetermined time. For example, the predetermined time is set to be one second approximately in advance.

### Example of First Learning

At step S107, the signal restoration system 1 performs the first learning. Hereinafter, learning performed with the first learning data as input data is referred to as the “first learning”.

As illustrated, after the integral value is calculated by the integral calculation, for example, processing at steps S108 to S110 is executed in parallel to steps S105 and S106. Note that, steps S108 to S110 do not necessarily need to be executed in parallel to steps S105 and S106.

### Example of Third Band-Pass Filter Processing

At step S108, separately from the first band-pass filter processing and the second band-pass filter processing, the signal restoration system 1 performs band-pass filter processing on the second signal. Hereinafter, the band-pass filter processing performed on the second signal separately from the second band-pass filter processing is referred to as “third band-pass filter processing”.

### Example of Peak Extraction

At step S109, the signal restoration system 1 extracts a peak from the signal provided with the third band-pass filter processing. The peak corresponds to a peak in an R wave.

### Example of Synchronization

At step S110, the signal restoration system 1 synchronizes the peak extracted at step S109 with a peak extracted at step S112 (the peak at step S112 will be described later in detail).

At step S110, the peak synchronized with the peak extracted at step S109 is, for example, a peak extracted through steps S121 and S122 below.

Steps S121 and S122 are executed in parallel to, for example, processing at steps S101 to S110. Note that, steps S121 and S122 do not necessarily need to be executed in parallel to steps S101 to S110.

### Example of ECG Signal Acquisition

At step S121, the signal restoration system 1 acquires an electrocardiogram signal (ECG signal). For example, the ECG signal is a signal generated by ECG, in other words, an electrocardiograph. Thus, the signal restoration system 1 is connected to, for example, the electrocardiograph or a device in which the ECG signal is stored, and acquires the ECG signal.

### Example of Peak Extraction

At step S122, the signal restoration system 1 extracts a peak from the ECG signal. The peak corresponds to a peak in an R wave.

For example, learning of a learning model as described below is performed through the “learning processing” as described above.

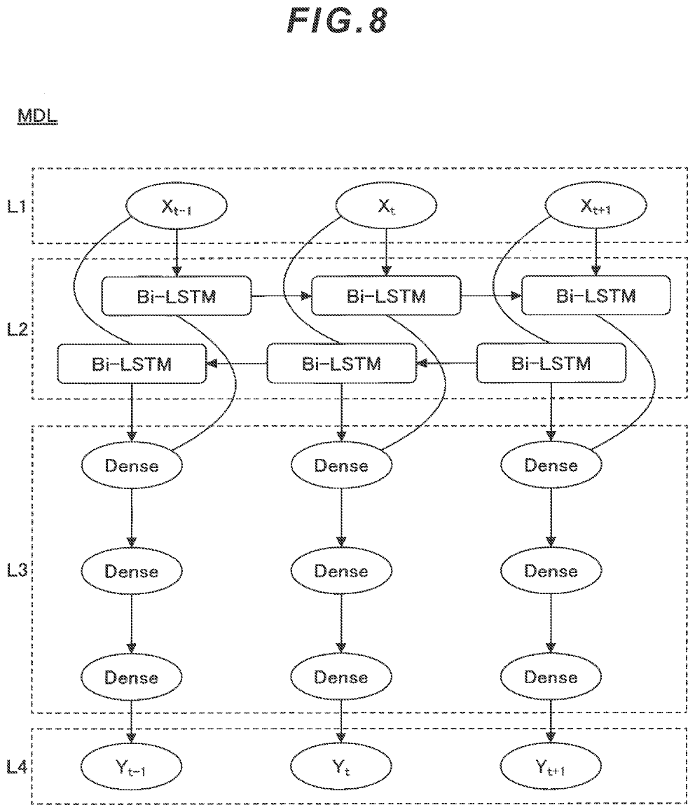

FIG. 8 is a diagram illustrating an exemplary network structure of a learning model. For example, a learning model MDL has a network structure including layers of an input L1, a multi-layer bidirectional long-short term memory (Bi-LSTM) L2, an affine layer L3, and an output L4.

The input L1 inputs data as “Xₜ₋₁”, “Xₜ”, and “Xₜ₊₁”. The output L4 outputs data as “yₜ₋₁”, “yₜ”, and “yₜ₊₁”. Note that, “t” represents an appearance time point of data. Thus, with “t” as a reference, “t−1” indicates data used in the previous cycle, and “t+1” indicates data used in the next cycle.

The multi-layer Bi-LSTM L2 is a two-layer Bi-LSTM. Time-series data can be processed with such a two-layer configuration of the multi-layer Bi-LSTM L2.

The affine layer L3 performs affine processing. Specifically, the affine processing is processing that, when a plurality of feature maps are generated by processing performed before the affine processing, associates each feature map with the output layer. The affine processing is also processing that determines, by an activation function or the like based on each feature map, whether a format set for final outputting corresponds to any output format set to the output layer in advance.

In this example, it is configured based on, for example, a sampling rate that the affine layer L3 includes three layers in the order of 512, 128, and 256.

The learning model MDL desirably has a network structure including an LSTM. In other words, the network structure of the learning model MDL desirably includes a RNN configuration.

For example, data as described below is input to the LSTM.

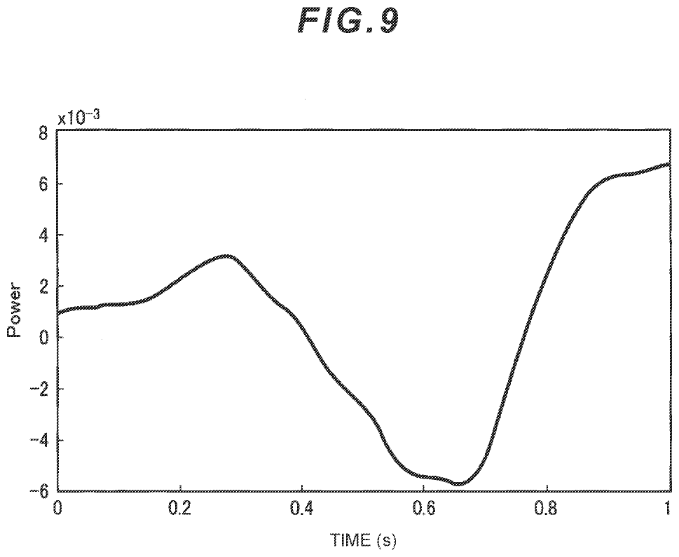

FIG. 9 is a diagram illustrating an exemplary input value. In the illustrated example, the horizontal axis represents time and the vertical axis represents the value of the integral value. In the illustrated example, the integral value has a width of one second. For example, in this manner, the integral value is input to the input side of the learning model MDL in the format of time-series data. Accordingly, for example, data as described below is output through processing at the multi-layer Bi-LSTM L2 and the affine layer L3.

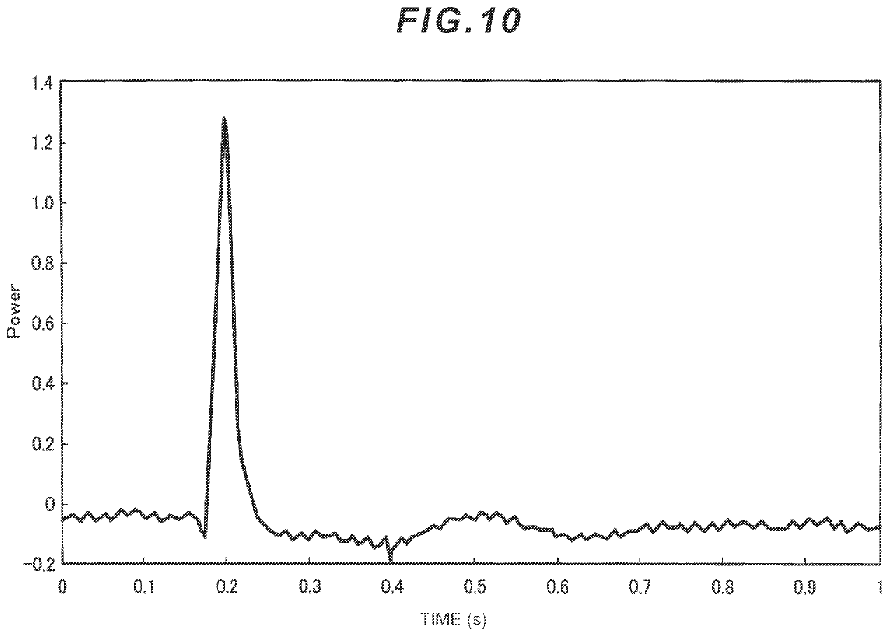

FIG. 10 is a diagram illustrating an exemplary output value. For example, the value is input to the output side of the learning model MDL in an ECG signal format with the width of one second as illustrated.

In the LSTM, processing is performed with a sigmoid function, a tank function, and the like. The processing is performed based on, for example, data input from a forget gate, an input gate, and an output gate. Thus, the input value as illustrated in FIG. 9 is input to the input gate, and the output value as illustrated in FIG. 10 is input to the output gate.

The multi-layer Bi-LSTM L2 desirably has a configuration (also referred to as “BLSTM” or the like) for performing processing in both backward and forward directions like the multi-layer Bi-LSTM L2 illustrated in FIG. 8. With such a configuration, high accuracy can be achieved.

For example, the first learning is performed by repeating the processing as described above. Such learning processing is performed to obtain the learning model by machine learning.

In this manner, when machine learning is performed with the LSTM, parameters of the learning model are set. The parameters are desirably optimized by machine learning. In this manner, a parameter setting unit configured to set parameters of a restored signal generator by machine learning using the LSTM is achieved. Hereinafter, the learning model for which learning is completed through the learning processing is referred to as the “learning-completed model”. After the learning-completed model is generated, the “execution processing” as described below is performed.

### Example of Second Heartbeat Signal Acquisition

At step S111, the signal restoration system 1 acquires a heartbeat signal. Hereinafter, a heartbeat signal for “actual measurement”, which is acquired separately from the “first heartbeat signal” is referred to as a “second heartbeat signal”. Thus, similarly to the first heartbeat signal, the second heartbeat signal is a signal representing heartbeat behavior and is IQ data generated by the Doppler radar 12.

### Exemplary Restored Signal Generation

At step S112, the signal restoration system 1 restores a signal representing heartbeat by using the learning-completed model. Hereinafter, the signal generated at step S112 is referred to as a “restored signal”.

Note that, similarly to the learning processing, the restored signal may be generated by processing such as steps S101 to S106. For example, the restored signal is generated as described below.

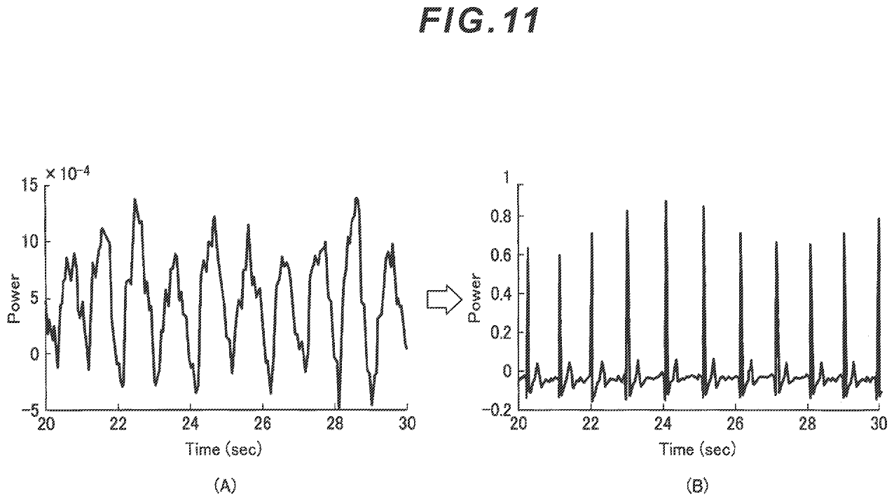

FIG. 11 is a diagram illustrating exemplary generation of a restored signal. For example, the second heartbeat signal as illustrated in FIG. 11(A) is acquired. When the “execution processing” is performed by using the learning-completed model, for example, a restored signal as illustrated in FIG. 11(B) is generated.

A restored signal is different from a heartbeat signal in that characteristics of a Q wave, an R wave, an S wave, a T wave, and the like in one period of heartbeat can be restored or enhanced as described below.

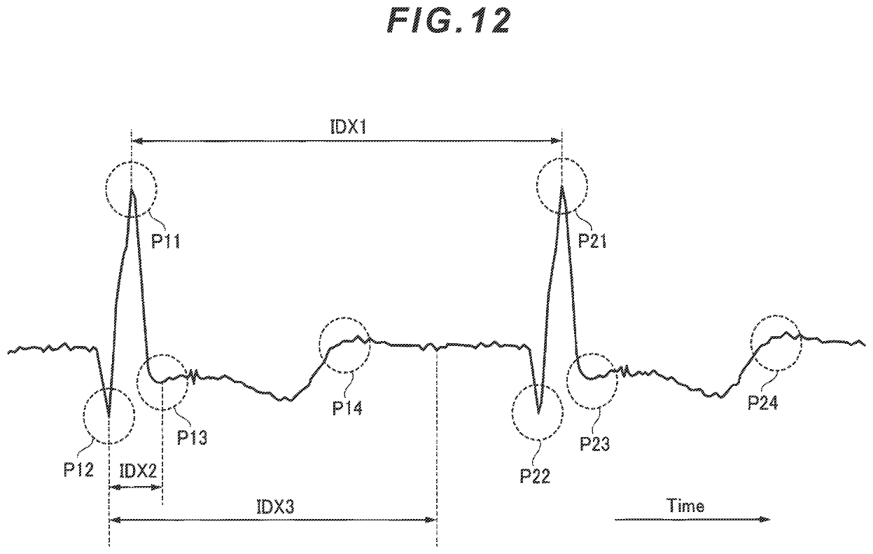

FIG. 12 is a diagram illustrating an example of a Q wave, an R wave, an S wave, and a T wave in one period of heartbeat. As illustrated, a restored signal is generated with which apexes such as an eleventh apex P11, a twelfth apex P12, a thirteenth apex P13, a fourteenth apex P14, a twenty-first apex P21, a twenty-second apex P22, a twenty-third apex P23, and a twenty-fourth apex P24 are restored or enhanced.

The eleventh apex P11 and the twenty-first apex P21 are apexes for detecting the R wave. When such apexes are clearly determined, for example, an R-R interval (RRI) can be accurately calculated. Specifically, a peak interval (hereinafter referred to as a “first indicator IDX1”) of the R wave in each period (in this example, the first period and the second period) can be calculated from the eleventh apex P11 and the twenty-first apex P21.

The first indicator IDX1 indicates one period of heartbeat. Typically, the first indicator IDX1 has a normal range of 600 ms to 1200 ms. Thus, when the first indicator IDX1 is accurately calculated, the period of heartbeat can be accurately understood.

The eleventh apex P11, the twelfth apex P12, and the thirteenth apex P13 are apexes for detecting the R wave, the Q wave, and the S wave. When such apexes are clearly determined, for example, a QRS interval can be accurately calculated. Thus, the interval (hereinafter referred to as a “second indicator IDX2”) of the Q wave to the S wave in one period can be calculated from the eleventh apex P11, the twelfth apex P12, and the thirteenth apex P13.

The second indicator IDX2 indicates the interval of systole of the cardiac ventricles. Typically, the second indicator IDX2 has a normal range of 60 ms to 100 ms. Thus, when the second indicator IDX2 is accurately calculated, systole of the cardiac ventricles can be accurately understood.

The twelfth apex P12 and the fourteenth apex P14 are apexes for detecting the Q wave and the T wave. When such apexes are clearly determined, for example, a QT interval can be accurately calculated. Thus, the interval (hereinafter, referred to as a “third indicator IDX3”) of the Q wave to the T wave in one period can be calculated from the twelfth apex P12 and the fourteenth apex P14.

The third indicator IDX3 indicates the interval of systole and diastole of the cardiac ventricles. Typically, the third indicator IDX3 has a normal range of 350 ms to 440 ms. Thus, when the third indicator IDX3 is accurately calculated, systole and diastole of the cardiac ventricles can be accurately understood.

As described above, indicators such as the first indicator IDX1, the second indicator IDX2, and the third indicator IDX3 can be accurately calculated by using the restored signal, and a health state can be accurately understood. Specifically, the indicators such as the first indicator IDX1, the second indicator IDX2, and the third indicator IDX3 are calculated and compared with their normal ranges to determine whether the normal ranges are exceeded. The case in which the normal ranges are exceeded corresponds to a case in which the heart or the like has anomaly. Thus, when the heart or the like has anomaly, the anomaly can be early found.

### Exemplary Filter Setting for Frequency Extracted in Band-Pass Filter Processing

In the learning processing and the execution processing, band-pass filter processing is desirably performed as preprocessing like the first band-pass filter processing and the second band-pass filter processing. The first band-pass filter processing and the second band-pass filter processing desirably have a relation as described below.

A frequency band excluded as an attenuation target is desirably set to be wider for the first band-pass filter processing than for the second band-pass filter processing.

For example, when a band-pass filter of 0.5 Hz to 2.0 Hz is applied to the integral value, a result as described below is obtained.

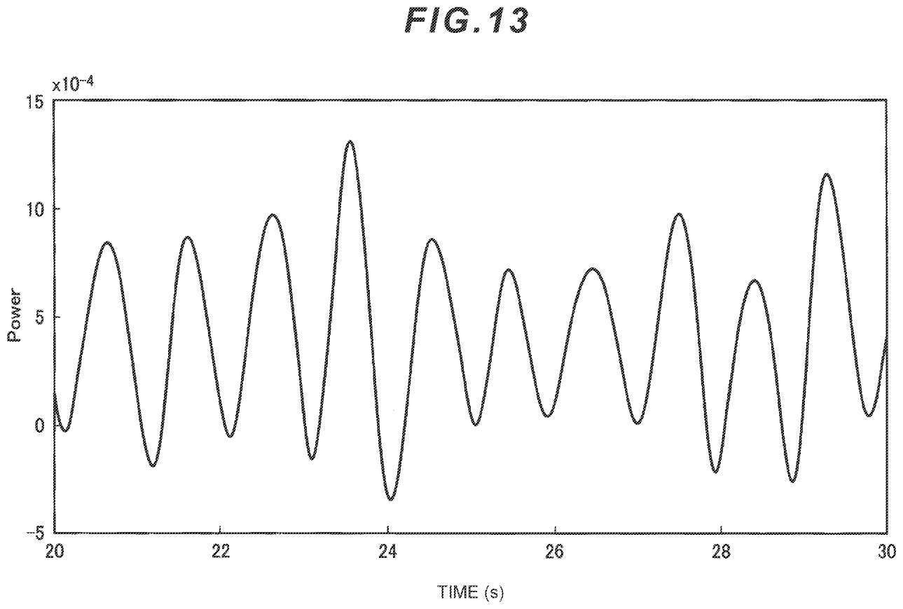

FIG. 13 is a diagram illustrating exemplary application of a band-pass filter of 0.5 Hz to 2.0 Hz. As illustrated, when the band-pass filter that extracts the frequency of 0.5 Hz to 2.0 Hz is applied, a waveform highly correlated with an R wave peak, in other words, a waveform correlated with systole of the heart is extracted.

When a band-pass filter that extracts the frequency of 0.5 Hz to 10.0 Hz is applied to the integral value, a result as described below is obtained.

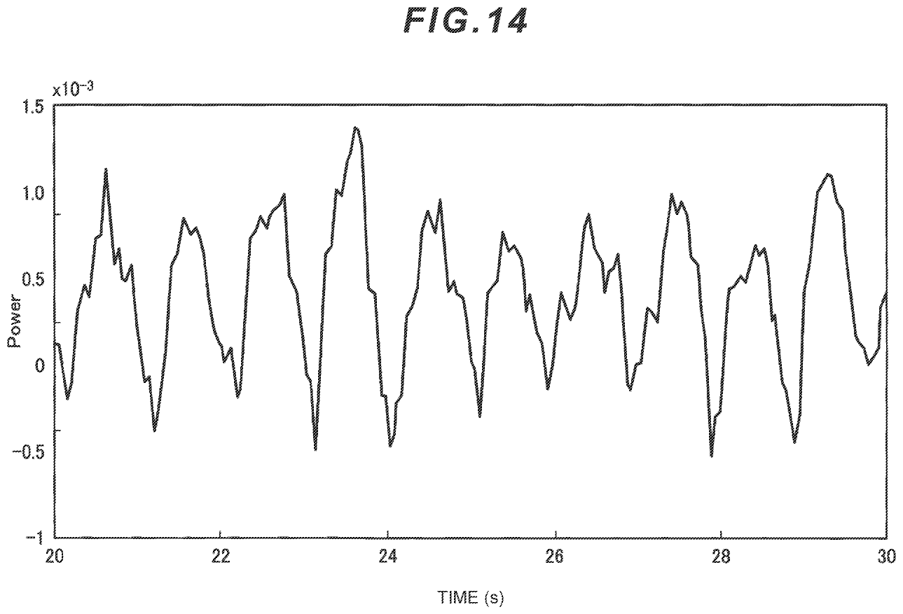

FIG. 14 is a diagram illustrating exemplary application of a band-pass filter of 0.5 Hz to 10.0 Hz. Comparison with the result illustrated in FIG. 13 indicates that the result illustrated in FIG. 14 includes a larger number of frequency components other than the R wave. Thus, when a band-pass filter is applied so that a frequency band of the waveform as illustrated in FIG. 14 is extracted, it is possible to accurately restore waveforms of the frequencies of the Q wave, the S wave, and the like other than the R wave from a restored signal and attenuate waveforms of frequencies of noise due to body motion and the like.

### Experiment Results

Results of an experiment with experiment specifications below are described.

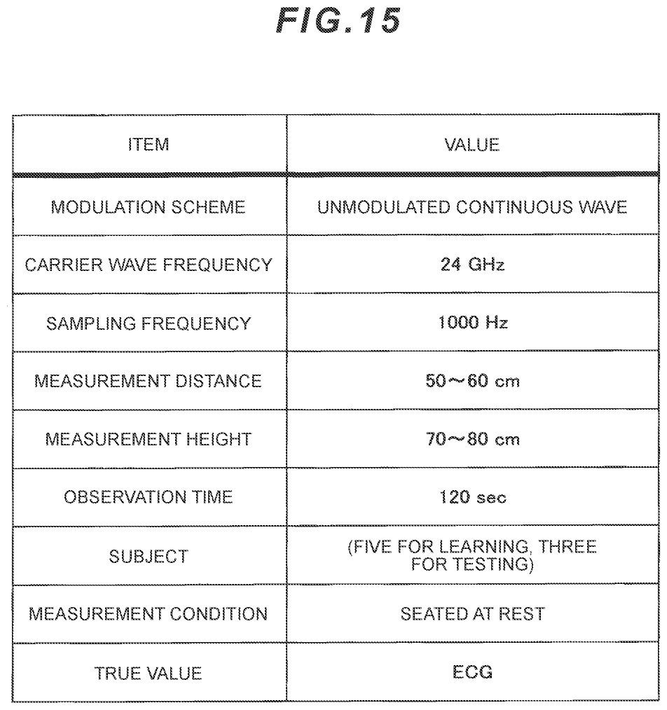

FIG. 15 is a table listing experiment specifications. The following describes a result of an experiment in which the waveform of “unmodulated continuous wave” having a frequency of “24 GHz” is sampled at “1000 Hz” as indicated in “modulation scheme”, “carrier wave frequency”, and “sampling frequency”. The same notations apply below.

The items “measurement distance” and “measurement height” indicate the distance between the Doppler radar 12 and the subject 2 and the height at which the Doppler radar 12 is installed in the experiment.

The item “observation time” indicates the time of heartbeat measurement.

The item “subject” indicates the number of target persons of “learning” and the number of target persons of “test”, in other words, the execution processing.

The item “measurement condition” indicates the posture of the subject in the experiment.

The item “true value” is “correct answer” data as a comparison target.

Evaluation indicators are a root mean square error (RMSE) calculated by Expression (7) below and an error average calculated by Expression (8) below.

$$\begin{matrix}[ Expression 7 ] & \\ R M S E = \sqrt{\frac{1}{N} {\sum }_{n = 1}^{N} {\vert X ( {t}_{n} ) - r ( {t}_{n} ) \vert}^{2}} & ( Expression 7 )\end{matrix}$$

$$N : Total number of peaks in observation time$$

$$n : Peak appearance time$$

$$X ( {t}_{n} ) : Peak interval obtained from ECG$$

$$r ( {t}_{n} ) : Estimated peak interval value$$

$${X}_{n} : Peak time obtained from ECG$$

$${r}_{n} : Estimated peak time$$

$$\begin{matrix}[ Expression 8 ] & \\ Error average = \frac{{\sum }_{n = 1}^{N} \vert {X}_{n} - {r}_{n} \vert}{N} & ( Expression 8 )\end{matrix}$$

where, the same variables as in (Expression 7) apply in this expression.

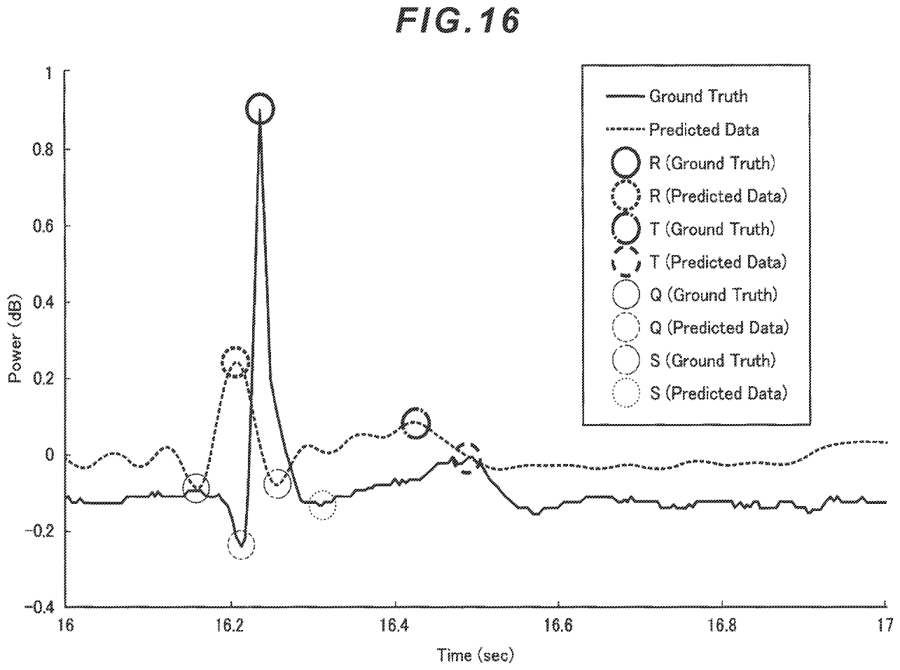

FIG. 16 is a diagram illustrating a comparative example in the experiment. When illustrated peaks of an R wave, a Q wave, an S wave, and a T wave are evaluated with the error average calculated by Expression (8) above, results as described below are obtained.

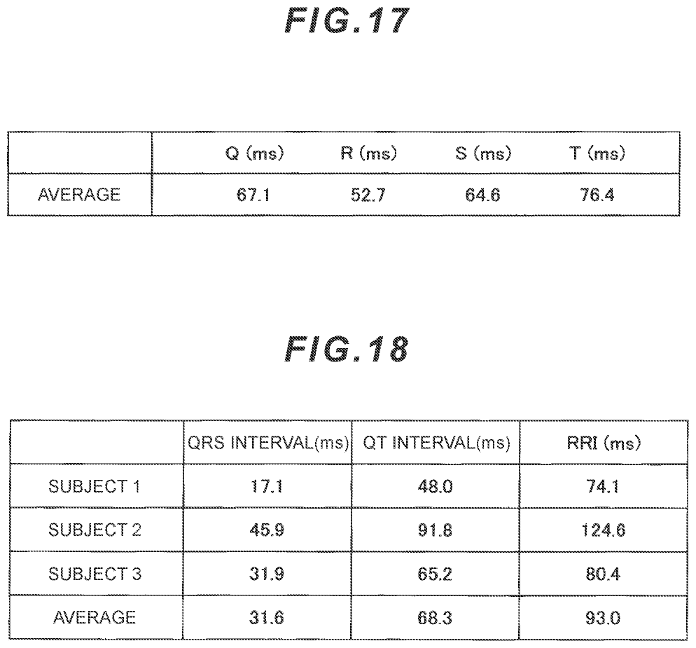

FIG. 17 is a diagram illustrating the error averages of the peaks. As illustrated, an experiment result with an error of “67.1 ms” in average from a true value, in other words, a signal measured by ECG was obtained for the peak indicating the Q wave.

An experiment result with an error of “52.7 ms” in average from a true value, in other words, a signal measured by ECG was obtained for the peak indicating the R wave.

An experiment result with an error of “64.6 ms” in average from a true value, in other words, a signal measured by ECG was obtained for the peak indicating the S wave.

An experiment result with an error of “76.4 ms” in average from a true value, in other words, a signal measured by ECG was obtained for the peak indicating the T wave.

In addition, the following results were obtained when the QRS interval, the QT interval, and the RRI were evaluated with the RMSE calculated by Expression (7) above as an indicator.

FIG. 18 is a diagram illustrating a comparative example of the QRS interval, the QT interval, and the RRI. Specifically, errors as described below occurred to the QRS interval and the QT interval.

As illustrated, the QRS interval had errors of “17.1 ms”, “45.9 ms”, and “31.9 ms” for three subjects and had an error of “31.6 ms” in average.

The QT interval had errors of “48.0 ms”, “91.8 ms”, and “65.2 ms” and had an error of “68.3 ms” in average.

The RRI had errors of “74.1 ms”, “124.6 ms”, and “80.4 ms” and had an error of “93.0 ms” in average.

Note that, when illustrated, the QRS interval and the QT interval are indicators as described below.

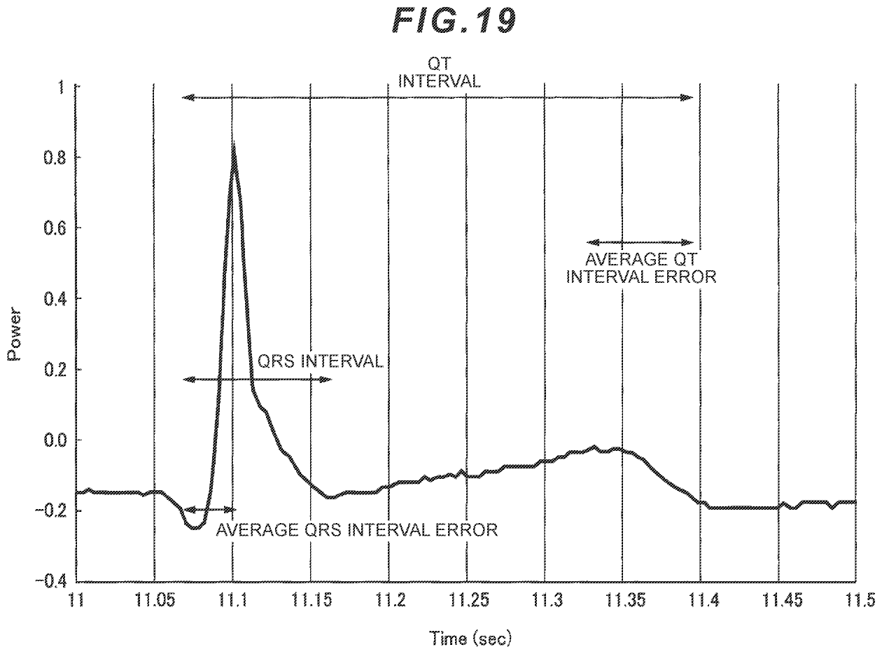

FIG. 19 is a diagram illustrating the errors of the QRS interval and the QT interval. In the drawing, “QRS interval” and “QT interval” indicate values calculated in the experiment. Errors indicated in “average” in FIG. 18 occurred to “average QRS interval error” and “average QT interval error”.

### Exemplary Functional Configuration

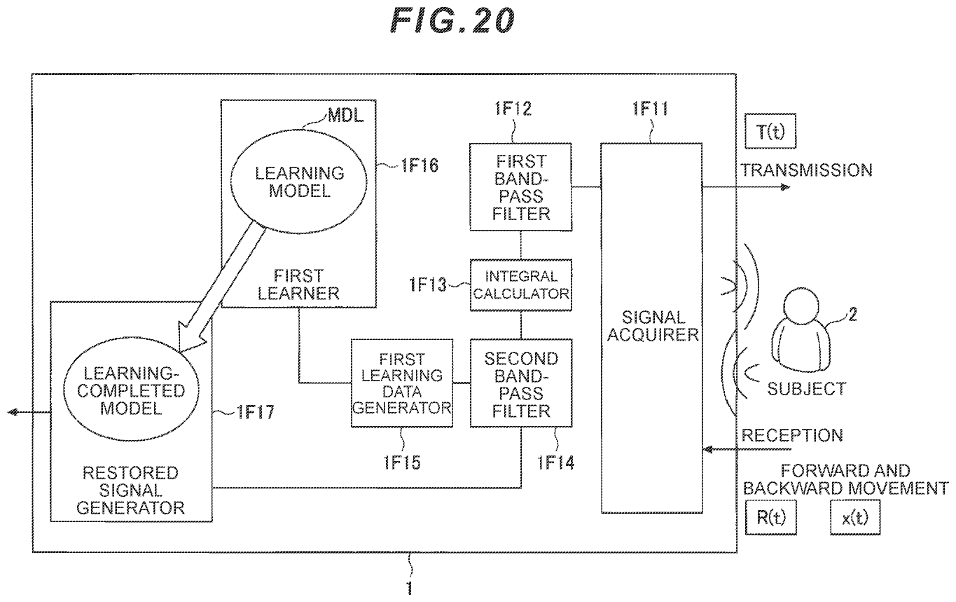

FIG. 20 is a diagram illustrating an exemplary functional configuration in the first embodiment. As illustrated, in a state in which the “learning processing” is performed, the signal restoration system 1 has a functional configuration including a signal acquirer 1F11, a first band-pass filter 1F12, an integral calculator 1F13, a second band-pass filter 1F14, a first learning data generator 1F15, and a first learner 1F16. In a state in which the “execution processing” is performed, the signal restoration system 1 has a functional configuration including the signal acquirer 1F11, the first band-pass filter 1F12, the integral calculator 1F13, the second band-pass filter 1F14, and a restored signal generator 1F17. The following description is made on, as an example, a state of a functional configuration including all functional configurations used in the “learning processing” and the “execution state”.

The signal acquirer 1F11 performs a signal acquisition procedure of acquiring heartbeat signals such as the first heartbeat signal and the second heartbeat signal. For example, the signal acquirer 1F11 is achieved by the Doppler radar 12 or the like.

The first band-pass filter 1F12 performs a first band-pass filter procedure of generating the first signal by performing the first band-pass filter processing on the first heartbeat signal. For example, the first band-pass filter 1F12 is achieved by the CPU 10H1 or the like.

The integral calculator 1F13 performs an integral calculation procedure of calculating the integral value by integrating the frequency intensity of heartbeat represented by the first signal. For example, the integral calculator 1F13 is achieved by the CPU 10H1 or the like.

The second band-pass filter 1F14 performs a second band-pass filter procedure of generating the third signal by performing the second band-pass filter processing on the second signal representing the integral value. For example, the second band-pass filter 1F14 is achieved by the CPU 10H1 or the like.

The first learning data generator 1F15 performs a first learning data generation procedure of generating the first learning data by dividing the third signal at intervals of a predetermined time. For example, the first learning data generator 1F15 is achieved by the CPU 10H1 or the like.

The first learner 1F16 performs a first learning procedure of inputting the first learning data and performing machine learning. For example, the first learner 1F16 is achieved by the CPU 10H1 or the like.

The restored signal generator 1F17 performs a restored signal generation procedure of acquiring the second heartbeat signal and generating the restored signal based on a learning-completed model generated by the machine learning. For example, the restored signal generator 1F17 is achieved by the CPU 10H1 or the like.

Machine learning of the learning model MDL is first performed through the “learning processing”. A “learning-completed model” is generated through such learning. Then, when the second heartbeat signal is acquired, the restored signal can be generated by using the learning-completed model.

As described in the above-described example, the signal restoration system 1 can generate a restored signal including the R wave, the Q wave, the S wave, and the T wave as illustrated FIG. 11(B). In other words, the signal restoration system 1 can generate a restored signal in which the R wave, the Q wave, the S wave, and the T wave can be easily observed. The indicators of the QRS interval, the QT interval, and the RRI can be accurately calculated by using such a restored signal. Thus, the signal restoration system 1 can accurately restore a signal representing heartbeat behavior, such as the restored signal.

The restored signal may be generated with enhancement of feature points such as peaks in the R wave, the Q wave, the S wave, and the T wave. In other words, the restored signal may be generated with enhancement of extreme values such as peaks in each wave.

### Second Embodiment

A second embodiment is achieved by, for example, an information processing device having the same entire configuration and the same hardware configuration as those of the first embodiment. Hereinafter, duplicate description of any feature of the first embodiment is omitted, and any different feature will be mainly described. The following example will be described with, as an exemplary signal generation system, the signal restoration system 1 having an entire configuration same as that in the first embodiment.

In the second embodiment, for example, blood pressure is estimated by detecting an aortic pulse wave as described below from a heartbeat signal acquired by the Doppler radar or the like.

The blood pressure indicates the pressure of blood flowing through blood vessels. For example, high blood pressure is potentially a main risk factor of a cardiac disease or the like, and the blood pressure is information that is important to monitor as living body information.

Conventionally, for example, auscultation by which the blood pressure is measured by a trained examiner listening Korotkov's sound by using a stethoscope has been known. In addition, for example, an oscillometric method of pressing an upper arm with a cuff and detecting pulsing has been known.

With the auscultation, it is difficult to easily perform measurement. Furthermore, with these methods, some subjects feel uncomfortable with constriction by a cuff. However, with a configuration using a heartbeat signal as in the present embodiment, contact with a subject is less, which can reduce subject's uncomfortable feeling due to contact.

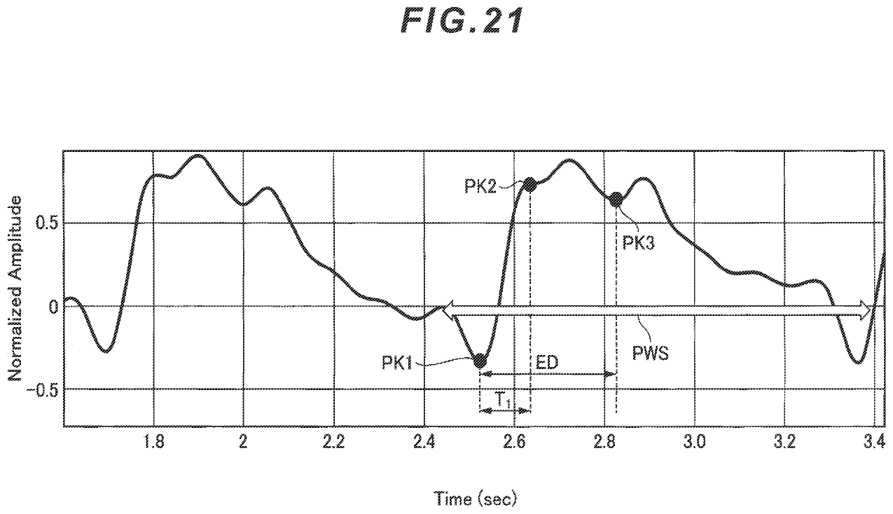

FIG. 21 is a diagram illustrating an example of an aortic pulse wave. For example, an aortic pulse wave signal PWS is a signal having an illustrated shape and a period of “2.5 sec” to “3.4 sec” in the drawing (time illustrated with an arrow in the drawing). The following description is made on the illustrated aortic pulse wave signal PWS as an example.

The aortic pulse wave signal PWS has a waveform attributable to motion of aorta. The aortic pulse wave signal PWS includes three characteristic points (in the drawing, a first peak point PK1, a second peak point PK2, and a third peak point PK3) illustrated as peaks in the drawing.

The first peak point PK1, the second peak point PK2, and the third peak point PK3 are extreme values of the aortic pulse wave signal PWS. Thus, the first peak point PK1, the second peak point PK2, and the third peak point PK3 can be specified by performing calculation that specifies extreme values through differential calculation (or difference calculation in a discrete case) of the aortic pulse wave signal PWS with respect to time.

The next peak of each of the first peak point PK1, the second peak point PK2, and the third peak point PK3 appears in a certain interval or later. Thus, for example, the second peak point PK2 is desirably detected in a subsequent time slot after elapse of a time in which the second peak point PK2 is expected to appear with respect to the first peak point PK1. In this manner, the interval in which peak points appear is constant to some extent due to properties of the aortic pulse wave signal PWS. A peak point that appears too close is likely to be noise. Thus, each peak point can be accurately detected by detecting the peak point in an interval range in which appearance is expected. Note that, the interval in which detection is performed is set in advance, for example.

The signal restoration system 1 first specifies a first interval (hereinafter represented by a variable “T₁”) and a second interval (hereinafter represented by a variable “ED”) based on peak points detected in this manner.

The variable “T₁” is the interval from rise of the pulse wave (in this example, the first peak point PK1 as a starting point) to a peak that appears right before a peak at a maximum amplitude (peak that appears on the mountain side; in this example, the second peak point PK2 as an end point).

The variable “ED” is the interval from rise of the pulse wave (in this example, the first peak point PK1 as a starting point) to a peak right after a peak at a maximum amplitude (peak that appears on the valley side; in this example, the third peak point PK3 as an end point).

These intervals are, for example, values written in “H. Zhao, et al., 2018 IEEE/MTT-S International Microwave Symposium, 20 Aug. 2018.”.

In this manner, once the aortic pulse wave signal PWS is generated, the values of intervals such as the first interval “T₁” and the second interval “ED” can be calculated by detecting peak points included in the aortic pulse wave signal PWS. In addition, once the aortic pulse wave signal PWS is generated, “PTT_{cf}” can be calculated based on the intervals through calculation as in Expression (9) below.

$$\begin{matrix}[ Expression 9 ] & \\ {PTT}_{cf} = \frac{ED - {T}_{1}}{2} & ( Expression 9 )\end{matrix}$$

There is a relation as described below between “PTT_{cf}” and the blood pressure.

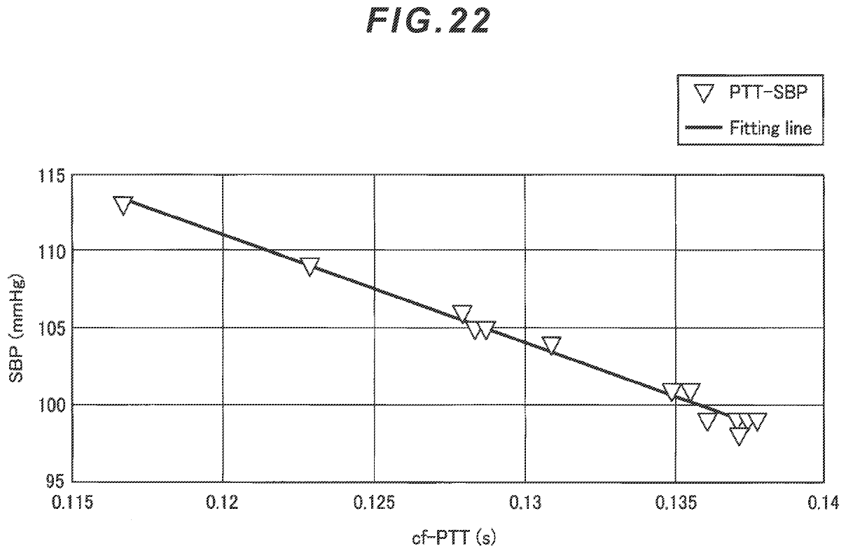

FIG. 22 is a diagram illustrating an exemplary relation between “PTT_{cf}” and the blood pressure. Specifically, the SBP and “PTT_{cf}” have a negative correlation therebetween. Thus, the relation is such that the blood pressure is higher as “PTT_{cf}” is shorter.

This relation can be expressed as Expression (10) below.

[Expression 10]

SBP=a×PTT_{cf}+b (Expression 10)

In Expression (10) above, “a” and “b” are values indicating the gradient and intercept of a linear function. Thus, once the parameters “a” and “b” are calculated, the linear function (straight line in FIG. 22) representing the relation between “PTT_{cf}” and the blood pressure can be specified based on Expression (10) above. Then, when “PTT_{cf}” is specified, the signal restoration system 1 can estimate the SBP, in other words, the blood pressure based on Expression (10) above.

This relation is written in, for example, “H. Zhao, et al., 2018 IEEE/MTT-S International Microwave Symposium, 20 Aug. 2018.”.

Accordingly, the signal restoration system 1 generates the aortic pulse wave signal PWS. The aortic pulse wave signal PWS is a signal as described below in an ideal environment, in other words, a noiseless environment.

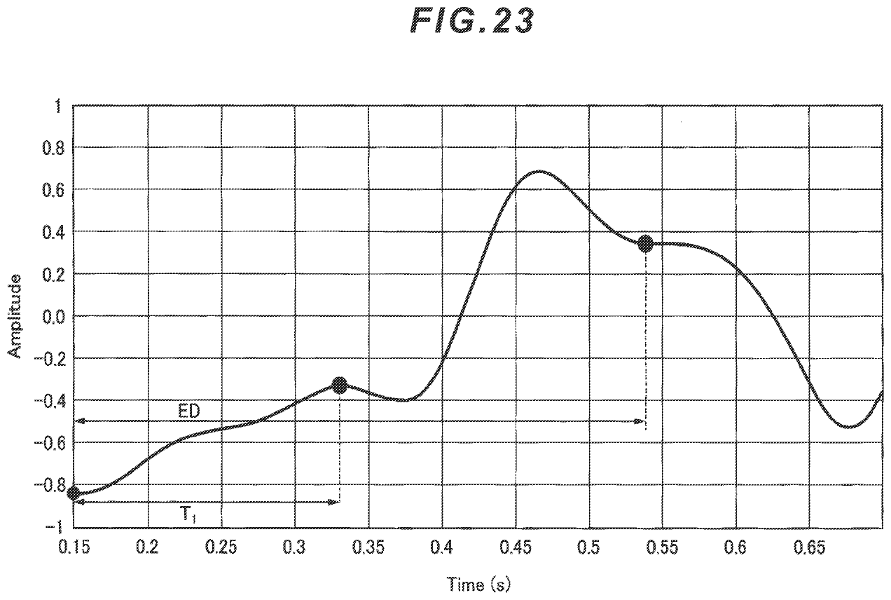

FIG. 23 is a diagram illustrating an example of the aortic pulse wave signal in an ideal state. For example, when the aortic pulse wave signal PWS is generated in a signal state that is close to the ideal state and with which the first interval “T₁” and the second interval “ED” can be easily calculated, in other words, with as little noise as possible as illustrated, the blood pressure can be accurately estimated based on Expressions (9) and (10) above.

In this manner, the aortic pulse wave signal PWS in the ideal state has a waveform with which the first interval “T₁” and the second interval “ED” can be calculated and “PTT_{cf}” and the blood pressure have a strong correlation therebetween. Note that, the strong correlation corresponds to, for example, a waveform having a correlation coefficient of “−0.7” or smaller. In particular, the aortic pulse wave signal PWS in the ideal state desirably has a waveform having a strong correlation coefficient of “−0.8” or smaller between “PTT_{cf}” and the blood pressure.

In reality, noise is included in signals acquired by the Doppler radar 12. Thus, the signal restoration system 1 inputs a heartbeat signal including noise and generates and outputs a signal with reduced noise as illustrated. The signal restoration system 1 generates the aortic pulse wave signal PWS and estimates the blood pressure through, for example, the entire processing as described below.

### Exemplary Entire Processing

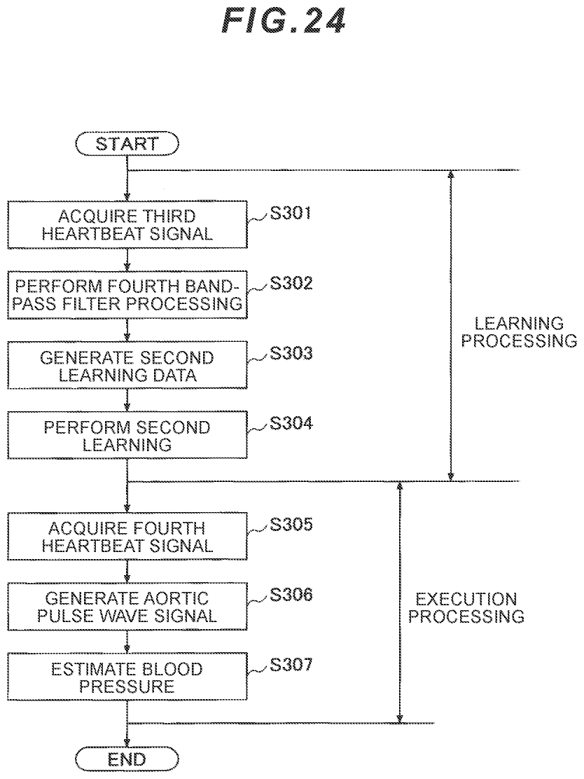

FIG. 24 is a diagram illustrating exemplary entire processing. The entire processing will be described below separately for the “learning processing” and the “execution processing”. Note that, the “learning processing” may be executed at any optional timing earlier than the “execution processing”. In other words, the “learning processing” and the “execution processing” do not necessarily need to be executed at continuous timings, and there may be a period before the “execution processing” is performed after the “learning processing”.

### Exemplary Acquisition of Third Heartbeat Signal

At step S301, the signal restoration system 1 acquires a heartbeat signal. Hereinafter, among heartbeat signals, a heartbeat signal used to generate “second learning data” as an example of second data to be described below is referred to as a “third heartbeat signal”. Accordingly, the third heartbeat signal is a signal that represents heartbeat behavior and on which learning data in machine learning is based, and is IQ data generated by the Doppler radar 12.

### Example of Fourth Band-Pass Filter Processing

At step S302, the signal restoration system 1 desirably performs band-pass filter processing on the third heartbeat signal. Hereinafter, the band-pass filter processing performed on the third heartbeat signal is referred to as “fourth band-pass filter processing”. A signal generated by performing the fourth band-pass filter processing on the third heartbeat signal, in other words, a signal generated by attenuating, through the fourth band-pass filter processing, a signal as noise included in the third heartbeat signal is referred to as a “fourth signal”.

The fourth band-pass filter processing is desirably set to extract the frequency of 0.5 Hz to 10.0 Hz approximately. More preferably, the fourth band-pass filter processing is desirably set to extract the frequency of 0.7 Hz to 7 Hz approximately.

### Exemplary Second Learning Data Generation

At step S303, the signal restoration system 1 generates the second learning data. For example, the second learning data is generated by dividing the fourth signal at intervals of a predetermined time, which is 0.8 seconds. Note that, the predetermined time is not limited to 0.8 seconds but may be, for example, 0.8±0.2 seconds approximately.

The second learning data is desirably generated, for example, as the aortic pulse wave signal PWS including noise, which is data input to the input side of the LSTM.

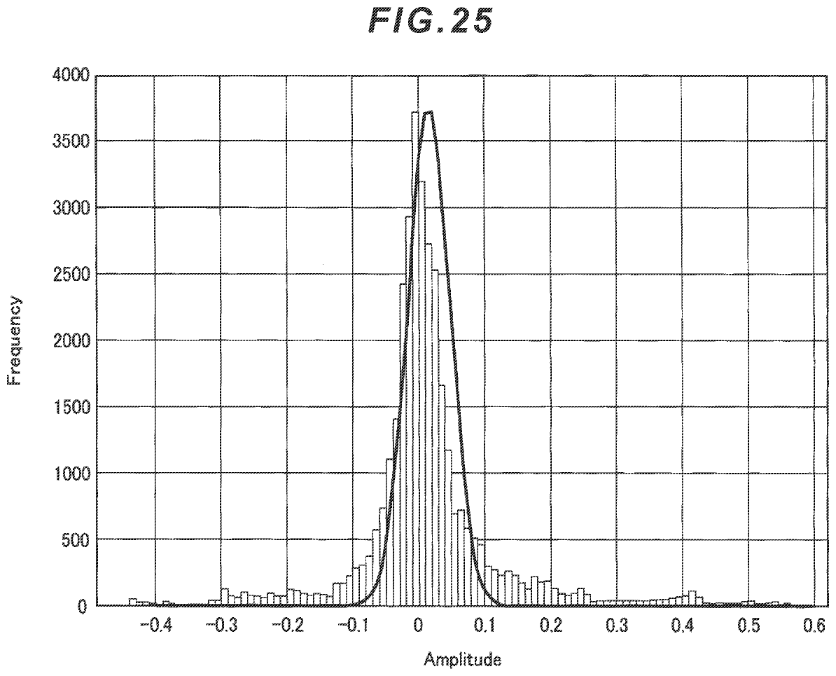

FIG. 25 is a diagram illustrating an example of a noise component used to generate the second learning data. Specifically, the second learning data may be generated by adding a noise component of an illustrated Gaussian distribution to the aortic pulse wave signal PWS in the ideal state. When the second learning data is generated by adding a noise component in this manner, an increased number of pieces of learning data can be obtained.

The aortic pulse wave signal PWS is likely to include noise having characteristics of a Gaussian distribution. Thus, when a learning model is subjected to learning so that noise of a Gaussian distribution can be attenuated, the noise can be accurately attenuated to extract the aortic pulse wave signal PWS.

Accordingly, data generated by adding a noise component of a Gaussian distribution is desirably used as the second learning data used for the input side.

Note that, in an environment in which noise that obeys a distribution different from a Gaussian distribution occurs, the learning model may be subjected to learning with the distribution taken into account. In this manner, when the learning model is subjected to learning in accordance with a noise distribution, noise can be accurately attenuated to extract the aortic pulse wave signal PWS.

Noise is modeled as described below, for example, and then added to the aortic pulse wave signal PWS in the ideal state.

First, the amplitude value of the aortic pulse wave signal PWS in the ideal state is specified at each time for each subject, and the average value thereof is calculated. Subsequently, for each subject, a noise component is calculated by subtracting the average value of the amplitude value of the aortic pulse wave signal PWS in the ideal state from the amplitude value of the aortic pulse wave signal PWS including noise. Subsequently, an S/N ratio (hereinafter referred to as “SNR”) is changed based on an assumed range of the SNR, and the noise component corresponding to the SNR is added to the aortic pulse wave signal PWS in the ideal state a plurality of times. In this manner, the second learning data is generated by adding the calculated noise component to the aortic pulse wave signal PWS in the ideal state. Accordingly, the second learning data is the aortic pulse wave signal PWS including the noise component, and the aortic pulse wave signal PWS in the ideal state.

### Example of Second Learning

At step S304, the signal restoration system 1 performs second learning. Hereinafter, learning with an LSTM for which the second learning data is input to the input side and the output side is referred to as “second learning”. Specifically, as the second learning data, the aortic pulse wave signal PWS including a noise component is used for the input side of a learning model of the LSTM, and the aortic pulse wave signal PWS in the ideal state is used for the output side of the learning model.

When the second learning is performed in this manner, a learning-completed model is generated to which the aortic pulse wave signal PWS including noise is input and that outputs the aortic pulse wave signal PWS in which the noise is attenuated.

### Exemplary Acquisition of Fourth Heartbeat Signal

At step S305, the signal restoration system 1 acquires a heartbeat signal. Hereinafter, a heartbeat signal for “actual measurement”, which is acquired separately from the “third heartbeat signal” is referred to as a “fourth heartbeat signal”. Thus, similarly to the fourth heartbeat signal, the third heartbeat signal is a signal representing heartbeat behavior and is IQ data generated by the Doppler radar 12.

### Exemplary Aortic Pulse Wave Signal Generation

At step S306, the signal restoration system 1 generates the aortic pulse wave signal by using the learning-completed model.

Note that, similarly to the learning processing, the processing at step S302 and the like may be performed to generate the aortic pulse wave signal.

### Example of Blood Pressure Estimation

At step S307, the signal restoration system 1 estimates the blood pressure. Specifically, the signal restoration system 1 calculates parameters such as the first interval “T₁”, the second interval “ED”, and “PTT_{cf}” based on the aortic pulse wave signal PWS generated at step S306. When the parameters are specified in this manner, the blood pressure can be estimated based on Expression (10) above.

### Experiment Results

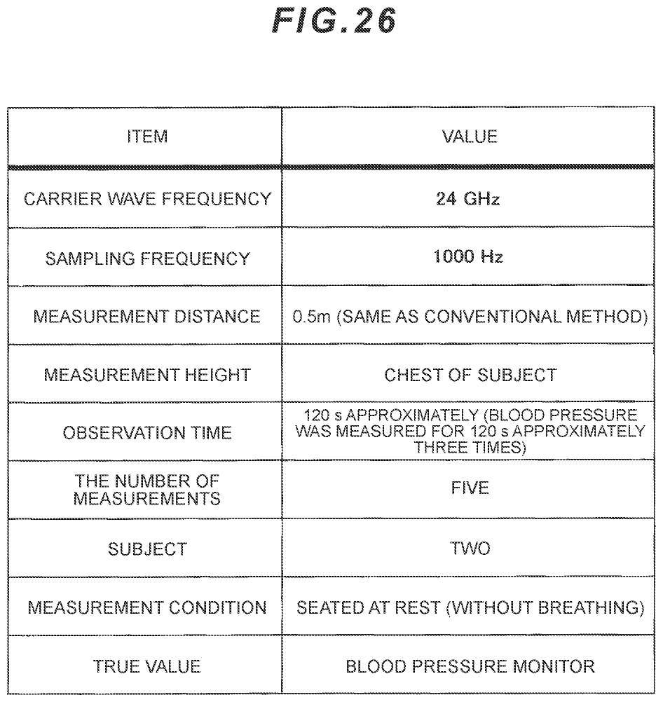

FIG. 26 is a table listing conditions under which learning data of the second embodiment is generated. Results of an experiment in which the second learning was performed by using the second learning data generated under the illustrated conditions will be described below. The item “true value” was obtained by “digital automatic blood pressure monitor HEM-907” (registered trademark) manufactured by OMRON Corporation.

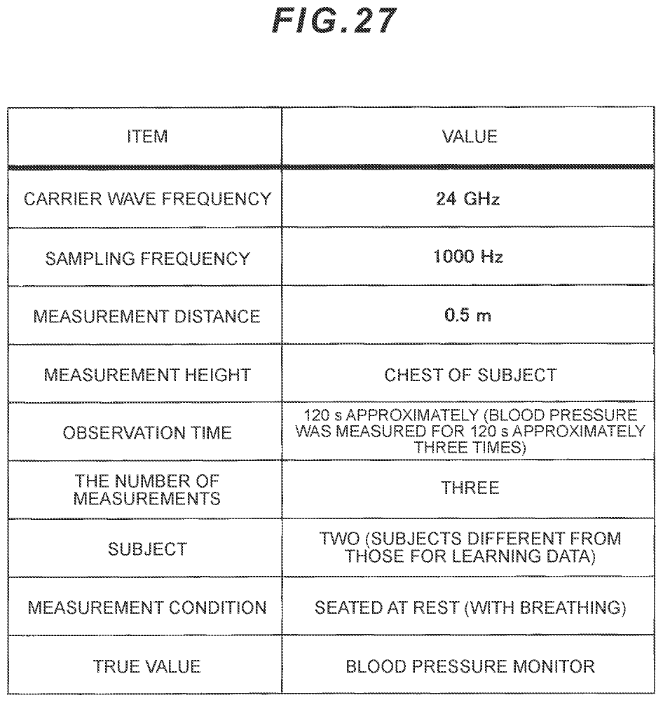

FIG. 27 is a table listing conditions under which data for execution of the second embodiment is generated. Results of an experiment in which the execution processing was performed by using the fourth heartbeat signal acquired under the illustrated conditions will be described below.

In the experiment, evaluation was performed based on experiment evaluation indicators (A) to (C) below.

(A) Ratio of a waveform for which the first interval “T₁” and the second interval “ED” cannot be calculated

(B) Coefficient of a correlation between the blood pressure of “true value” and “PTT_{cf}”

(C) Error between the blood pressure of “true value” and blood pressure indicated by an estimation result

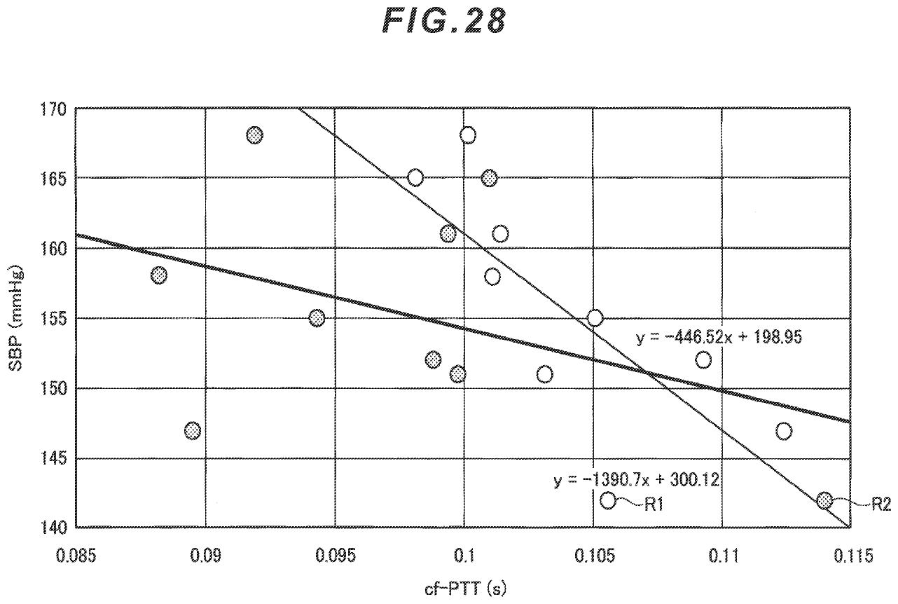

FIG. 28 is a scatter diagram of the blood pressure and “PTT_{cf}” and is a diagram illustrating approximate straight lines thereof. The drawing illustrates an experiment result of one of a plurality of subjects in the experiment. In the drawing, a comparison experiment result (hereinafter referred to as a “comparative example R1”) and an experiment result according to the present embodiment (hereinafter referred to as a “proposed method R2”) are plotted.

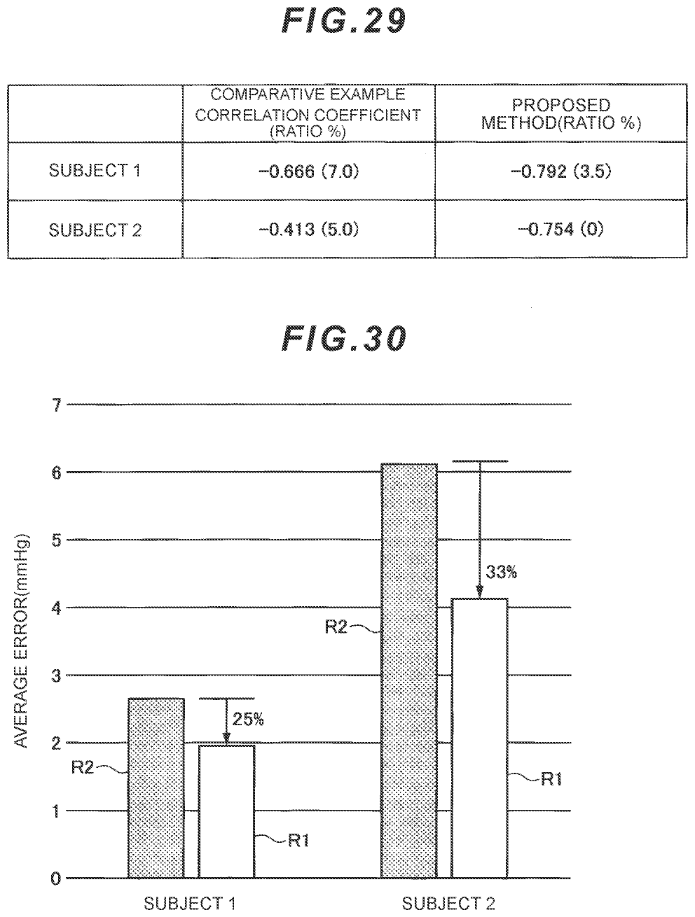

FIG. 29 is a diagram illustrating a result calculation of the ratio of a waveform for which the first interval “T₁” and the second interval “ED” cannot be calculated and a calculation result of the correlation coefficient. In the table, “comparative example” denotes experiment results obtained by a method corresponding to the comparative example R1 in FIG. 28. In addition, “proposed method” denotes experiment results obtained by a method corresponding to the proposed method R2 in FIG. 28.

FIG. 29 illustrates experiment results for two subjects, namely, “subject 1” and “subject 2”. Correlation coefficients (values expressed as negative values) in the drawing are experiment results of (B) the coefficient of correlation between the blood pressure of “true value” and “PTT_{cf}”. The values of “ratio” in parentheses in the drawing are experiment results of (A) the ratio of a waveform for which the first interval “T₁” and the second interval “ED” cannot be calculated.

As illustrated, (A) the ratio of a waveform for which the first interval “T₁” and the second interval “ED” cannot be calculated was lower for the proposed method for both subjects. Thus, the proposed method is more likely to generate a waveform for which parameters such as the first interval “T₁” and the second interval “ED” can be calculated.

(B) The coefficient of correlation between the blood pressure of “true value” and “PTT_{cf}” indicates a higher correlation for the proposed method for both subjects.

FIG. 30 is a diagram illustrating experiment results of the error between the blood pressure of “true value” and the blood pressure indicated by an estimation result.

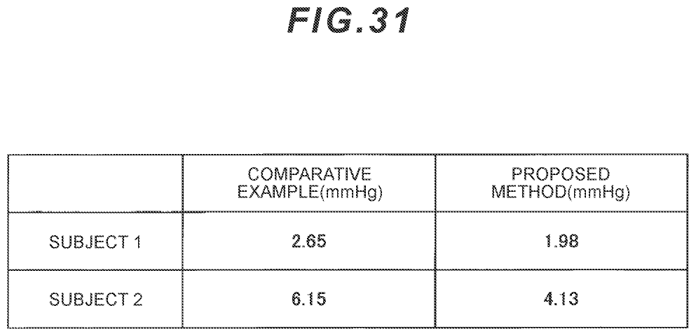

FIG. 31 is a diagram illustrating experiment results of the error between the blood pressure of “true value” and the blood pressure indicated by an estimation result.

In FIGS. 30 and 31, “comparative example” denotes experiment results obtained by the method corresponding to the comparative example R1 in FIG. 28. In addition, “proposed method” denotes experiment results obtained by the method corresponding to the proposed method R2 in FIG. 28.

As illustrated, the error for the proposed method was smaller by “25%” approximately for “subject 1” than for the comparative example. Similarly, the error for the proposed method was smaller by “33%” approximately for “subject 2” than for the comparative example. In this manner, the proposed method estimated the blood pressure with a smaller (C) error between the blood pressure of “true value” and the blood pressure indicated by an estimation result than the comparative example.

### Exemplary Functional Configuration

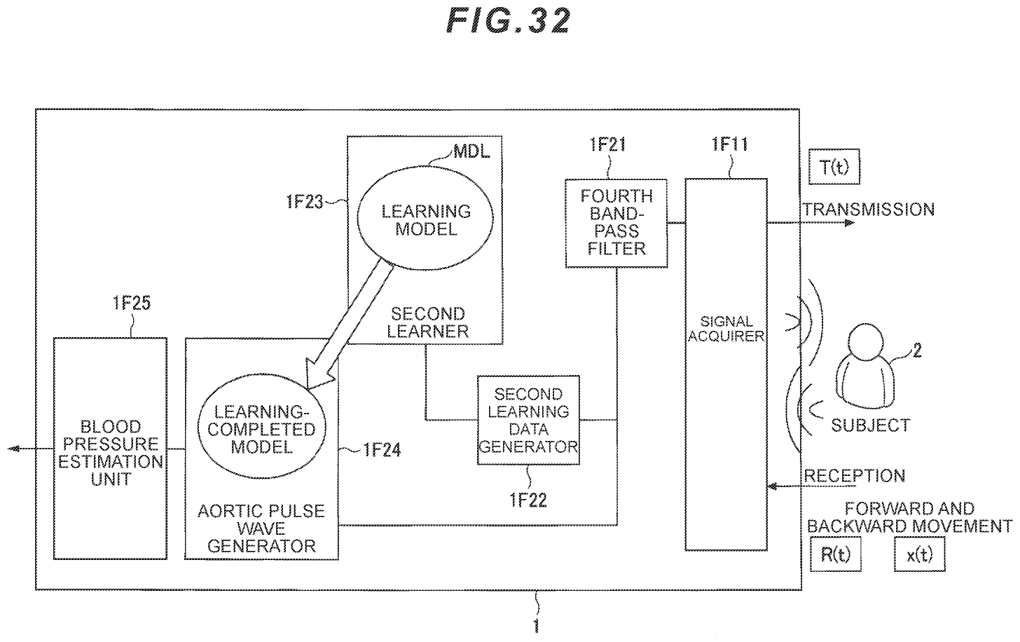

FIG. 32 is a diagram illustrating an exemplary functional configuration in the second embodiment. As illustrated, in a state in which the “learning processing” is performed, the signal restoration system 1 has a functional configuration including the signal acquirer 1F11, a fourth band-pass filter 1F21, a second learning data generator 1F22, and a second learner 1F23. In a state in which the “execution processing” is performed, the signal restoration system 1 has a functional configuration including the signal acquirer 1F11, the fourth band-pass filter 1F21, an aortic pulse wave generator 1F24, and a blood pressure estimation unit 1F25. The following description is made on, as an example, a state of a functional configuration including all functional configurations used in the “learning processing” and the “execution state”.

The signal acquirer 1F11 performs a signal acquisition procedure of acquiring heartbeat signals such as the third heartbeat signal and the fourth heartbeat signal. For example, the signal acquirer 1F11 is achieved by the Doppler radar 12 or the like.

The fourth band-pass filter 1F21 performs a fourth band-pass filter procedure of generating the fourth signal by performing the fourth band-pass filter processing on the third heartbeat signal. For example, the fourth band-pass filter 1F21 is achieved by the CPU 10H1 or the like.

The second learning data generator 1F22 performs a second learning data generation procedure of generating the second learning data by dividing the fourth signal at intervals of a predetermined time. For example, the second learning data generator 1F22 is achieved by the CPU 10H1 or the like.

The second learner 1F23 performs a second learning procedure of inputting the second learning data and performing machine learning. For example, the second learner 1F23 is achieved by the CPU 10H1 or the like.

The aortic pulse wave generator 1F24 performs an aortic pulse wave generation procedure of acquiring the fourth heartbeat signal and generating, based on a learning-completed model generated by the machine learning, an aortic pulse wave signal including aortic pulse wave or obtained by enhancing the aortic pulse wave. For example, the aortic pulse wave generator 1F24 is achieved by the CPU 10H1 or the like.

The blood pressure estimation unit 1F25 performs a blood pressure estimation procedure of estimating the blood pressure based on a parameter represented by the aortic pulse wave signal. For example, the blood pressure estimation unit 1F25 is achieved by the CPU 10H1 or the like.

Machine learning of the learning model MDL is first performed through the “learning processing”. A “learning-completed model” is generated through such learning. Then, when the fourth heartbeat signal is acquired, the aortic pulse wave signal can be generated by using the learning-completed model. When the aortic pulse wave signal is obtained, parameters such as the first interval “T₁”, the second interval “ED”, and “PTT_{cf}” are specified and the blood pressure can be estimated based on Expression (10) above.

With the above-described configuration, the signal restoration system 1 can generate the aortic pulse wave signal and estimate the blood pressure.

When generating the aortic pulse wave signal, the aortic pulse wave generator 1F24 may generate the aortic pulse wave signal in an enhancing manner. Specifically, with the configuration as described above, parameters such as the first interval “T₁” and the second interval “ED” are calculated based on the aortic pulse wave signal. In the calculation, the parameters can be more accurately calculated when extreme values of the aortic pulse wave signal, in other words, the first peak point PK1, the second peak point PK2, the third peak point PK3, and the like in FIG. 21 are clear. Thus, the aortic pulse wave generator 1F24 may further perform processing such as fabrication of the waveform to enhance the extreme values. In addition, for example, whether each extreme value is convex downward or upward may be calculated with the second-order differential or the like.

### Exemplary IQ Data Measured by Doppler Radar

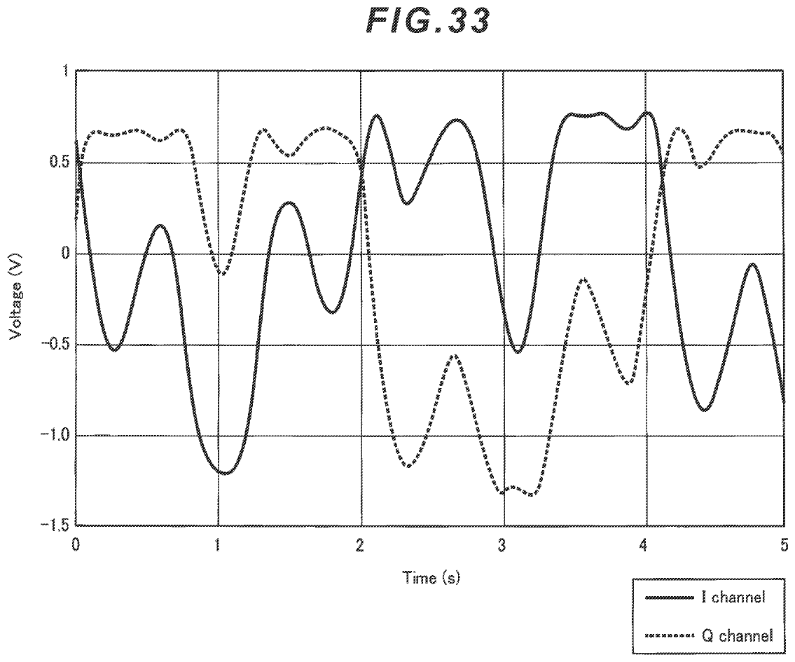

FIG. 33 is an example of IQ data measured by the Doppler radar. For example, the Doppler radar 12 outputs illustrated signals. Then, a heartbeat signal is obtained by calculating the arctan(Q/I).

By irradiating a moving object with electric wave, the Doppler radar 12 can measure motion of the object based on the Doppler effect that the frequency of reflection wave changes. Thus, it is desirable to have a configuration with which motion of a subject can be measured in this non-contact manner.

### Third Embodiment

A third embodiment is achieved by, for example, an information processing device having the same entire configuration and the same hardware configuration as those of the first embodiment. Hereinafter, duplicate description of any feature of the first embodiment is omitted, and any different feature will be mainly described. The following example will be described with, as an exemplary signal generation system, the signal restoration system 1 having an entire configuration same as that in the first embodiment.

In the third embodiment, for example, a Doppler signal as indicated by Expression (11) below is acquired by the Doppler radar or the like and a heartbeat signal is reconstructed.

[Expression 11]

I(t)+jQ(t) (Expression 11)

Then, for example, processing as described below is provided to the Doppler signal indicated by Expression (11) above.

First, band-pass filter processing is desirably performed with a cutoff frequency set to 0.5 Hz and 2.0 Hz.

Secondly, SIFT is performed with a window size of “256 ms” or “512 ms” and a step size of “5 ms” to “50 ms” approximately.

Thirdly, processing such as restoration is performed based on an LSTM. Specifically, a heartbeat signal is generated from a spectrogram by using the LSTM.

The LSTM is an exemplary deep layer learning method by which a long-period dependency relation of a signal in the time domain can be learned. When the LSTM has a configuration (Bi-LSTM) for performing bidirectional processing as described in the above-described example, the long-period dependency relation of a signal can be learned in the two directions of forward and backward directions of time.

The spectrogram is divided at intervals of several seconds, and power of a frequency band generated by a spectrogram attributable to heartbeat is input as input data to the LSTM.

In addition, a signal from which heartbeat behavior can be easily detected is desirably used as output data for the LSTM. For example, a signal generated by performing filter processing on the ECG signal or the ECG signal is desirably used.

A learning model desirably includes, for example, three layers of an input layer, a Bi-LSTM layer, and a regression layer. When the Bi-LSTM layer and the regression layer have multi-layer configurations, a signal in which heartbeat behavior is restored can be generated based on a more detailed characteristic amount.

Overlearning is more likely to occur as a network structure is more complicated. A structure of three layers approximately is a simple structure and thus is desirable.

The number of hidden layers and the step size of the Bi-LSTM are desirably a value with a power-of-two input data length and “64” to “256” approximately.

A loss function is a difference from the first embodiment as described below.

The loss function is desirably a function that uses a correlation coefficient “coef” so that learning of a learning model is performed to have a high correlation between an output waveform and a true value.

Specifically, the loss function is set to, for example, a function as in Expression (12) below.

[Expression 12]

loss=1−coef (Expression 12)

With the configuration as described above, for example, results as described below are obtained.

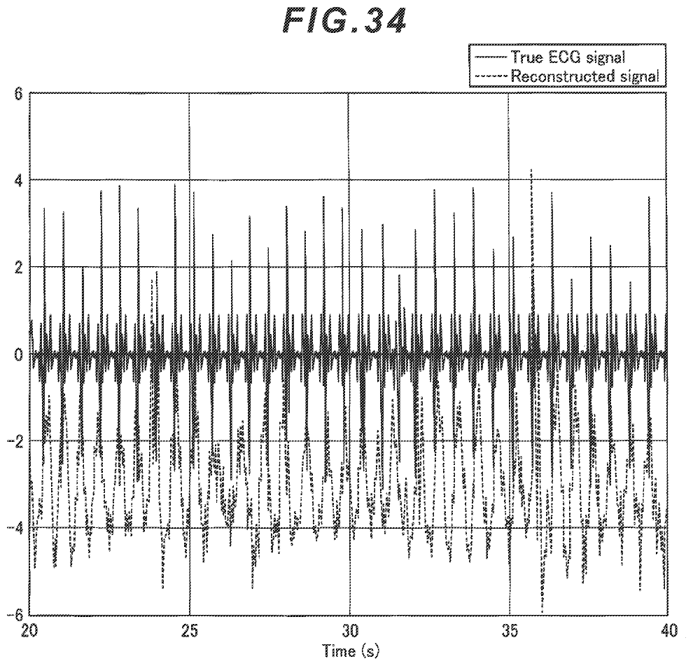

FIG. 34 is a diagram illustrating an example of a result of comparison with the ECG signal. In this experiment, a subject was in a supine position on a bed.

The vertical axis represents voltage. The horizontal axis represents time.

In the illustrated evaluation, the window size and step size of SIFT were set to “512 ms” and “25 ms”, respectively, with consideration of a calculation amount. Then, band-pass filter processing was performed to have [−20, −8.0] Hz and [8.0, 20] Hz as frequency bands used for input. The illustrated signals are exemplary output signals by a produced deep learning model. The line denoted by “True ECG signal” represents the ECG signal. The line denoted by “Reconstructed signal” is an output signal by the learning model (in other words, output from an LSTM). In this manner, peaks corresponding to peaks of the ECG signal can be observed with the output signals.

Comparison between RRIs calculated with the ECG signal and the output signal by the learning model obtains results as described below. Note that, in drawings described below, the ECG signal and the output by the learning model are normalized so that a peak correspondence relation can be easily observed (the vertical axis represents a normalization value).

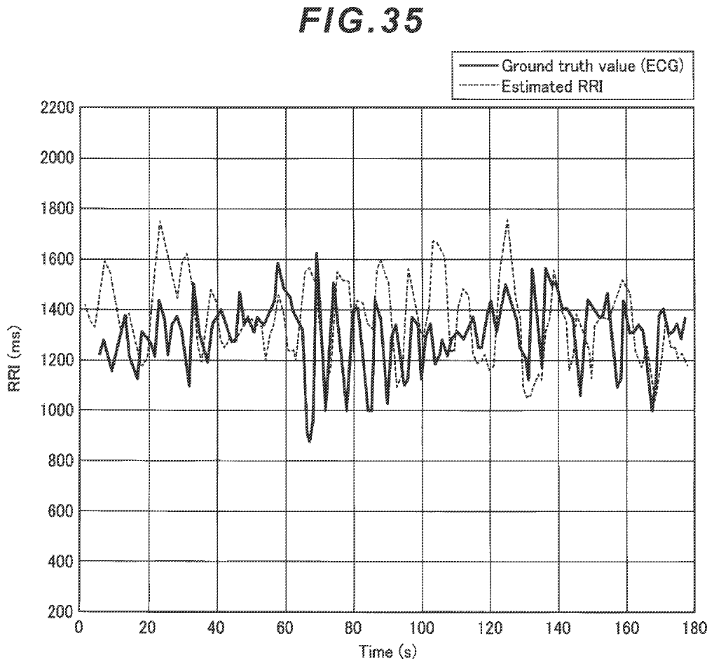

FIG. 35 is a diagram illustrating a first estimation result.

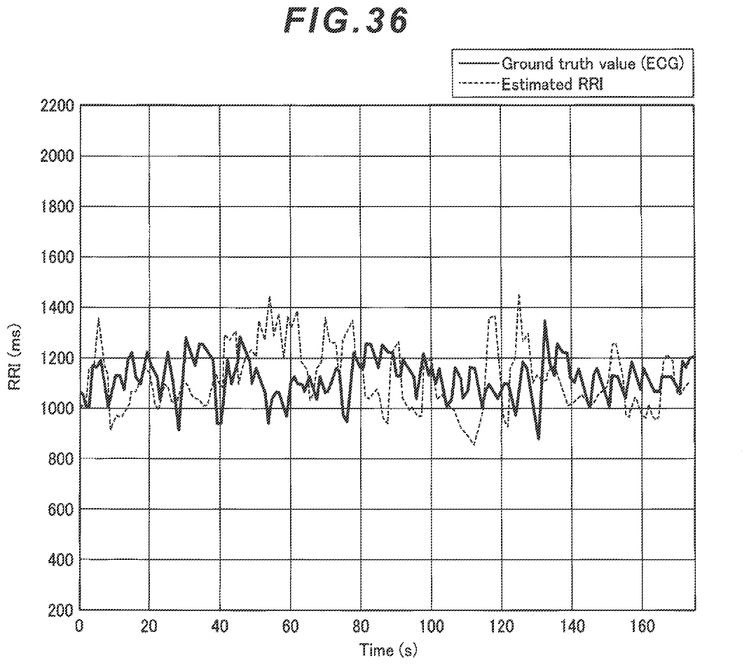

FIG. 36 is a diagram illustrating a second estimation result.

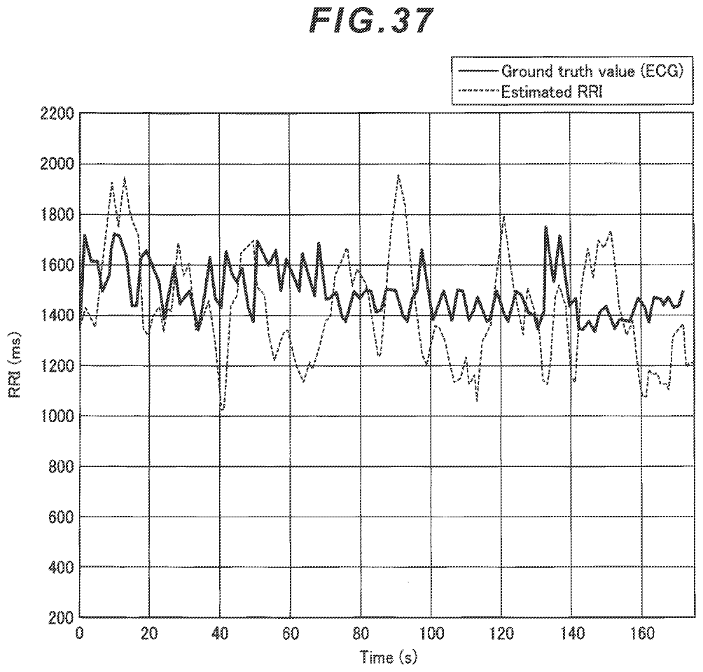

FIG. 37 is a diagram illustrating a third estimation result.

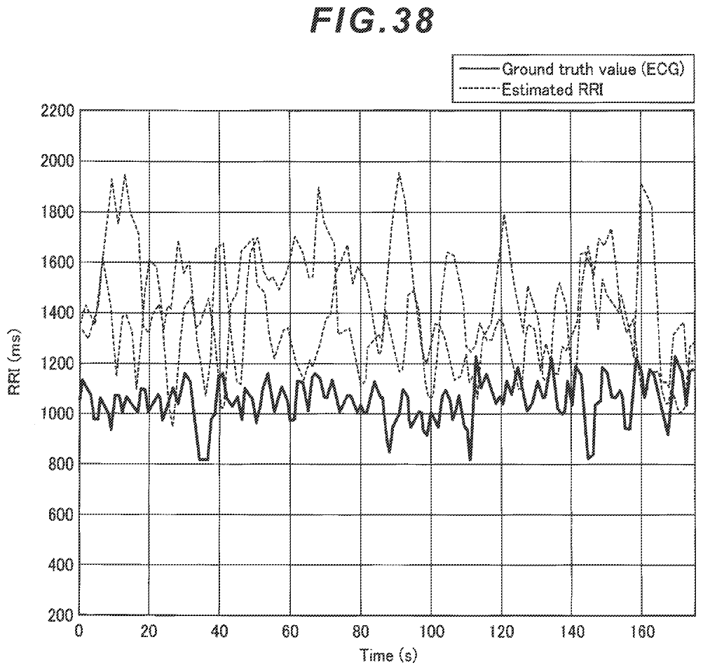

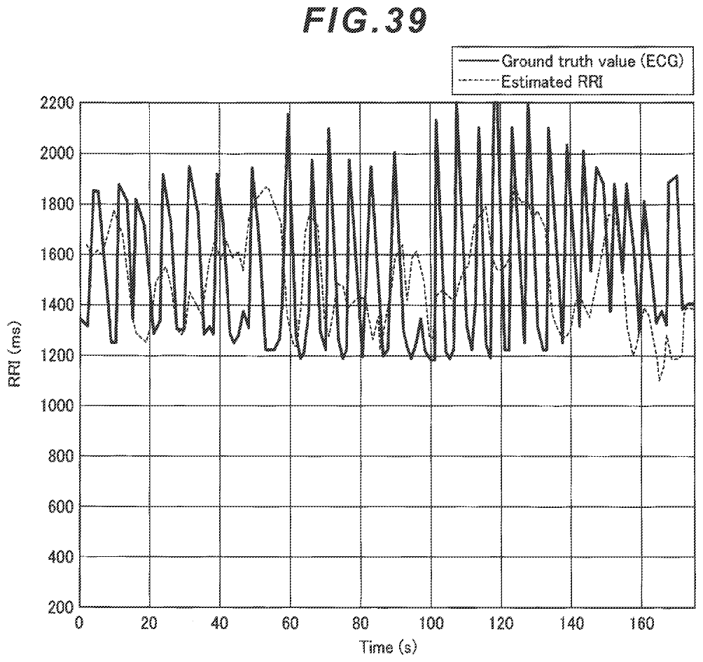

FIG. 38 is a diagram illustrating a fourth estimation result.

FIG. 39 is a diagram illustrating a fifth estimation result.

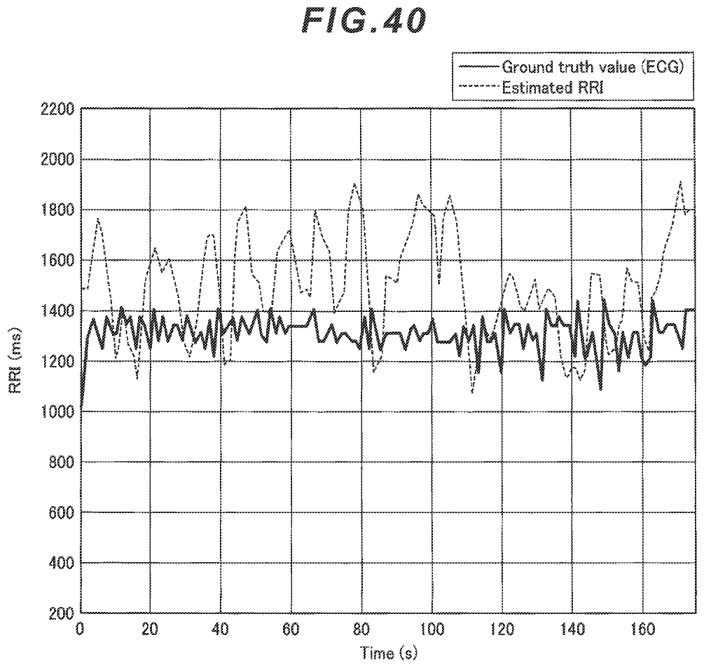

FIG. 40 is a diagram illustrating a sixth estimation result.

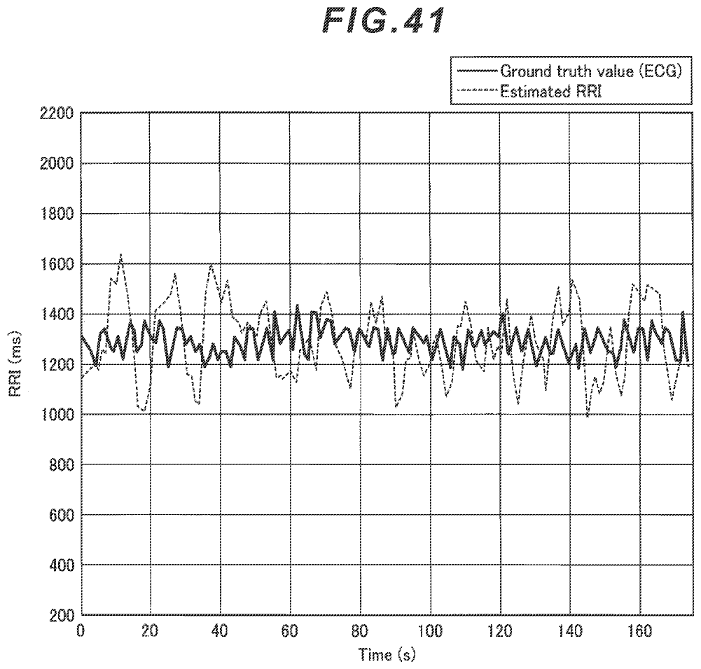

FIG. 41 is a diagram illustrating a seventh estimation result.

Subjects were different among the first to seventh estimation results. Note that, the subjects in the first to seventh estimation results were seated in a rest state.

As illustrated, characteristics close to those of ECG can be obtained with the present embodiment (“Estimated RRI” in the drawings).

The present embodiment has a configuration having a long input time width and including a plurality of peaks. With the configuration, processing such as peak association in the first embodiment is unnecessary.

### Modifications

Constituent components described in the first and second embodiments may be combined. For example, heartbeat signal may be acquired by a signal restoration system having both learning-completed models in the first and second embodiments and used for both models. In this manner, the present invention is also applicable to a configuration in which the constituent components of the first and second embodiments are partially used in common.

Signals are desirably generated at intervals equal to one period of heartbeat or the like. However, two or more periods may be included in one piece of data.

### Embodiments of Learning-Completed Model

A learning-completed model for causing a computer to function to acquire a second heartbeat signal and generate a restored signal representing heartbeat behavior,

the learning-completed model having a network structure including
an input layer,
an LSTM layer including an LSTM,
an affine layer, and
an output layer,

the learning-completed model being subjected to learning when a signal restoration system
acquires a first heartbeat signal representing heartbeat behavior,
generates a first signal by performing first band-pass filter processing on the first heartbeat signal,
calculates an integral value by integrating frequency intensity of the heartbeat represented by the first signal,
generates a third signal by performing second band-pass filter processing on a second signal representing the integral value with respect to time,
generates first learning data by dividing the third signal at intervals of a predetermined time, and
inputs the first learning data,

wherein the learning-completed model may cause the computer to function to
calculate an integral value based on the second heartbeat signal,
input the integral value to the input layer of the learning-completed model, and
generate the restored signal.

A learning-completed model for causing a computer to function to acquire a fourth heartbeat signal, generate an aortic pulse wave signal including aortic pulse wave or obtained by enhancing the aortic pulse wave, and estimate blood pressure based on a parameter represented by the aortic pulse wave signal,

the learning-completed model having a network structure including
an input layer,
an LSTM layer including an LSTM,
an affine layer, and
an output layer,

the learning-completed model being subjected to learning when a signal generation system
acquires a third heartbeat signal representing heartbeat behavior,
generates a fourth signal by performing fourth band-pass filter processing on the third heartbeat signal,
generates second learning data by dividing the fourth signal at intervals of a predetermined time, and
inputs the second learning data,

wherein the learning-completed model may cause the computer to function to
generate an aortic pulse wave signal including the aortic pulse wave or obtained by enhancing the aortic pulse wave when the fourth heartbeat signal is input to the learning-completed model, and
estimate blood pressure based on the aortic pulse wave signal.

A learning-completed model is used as part of AI software. Accordingly, the learning-completed model is a computer program. Thus, the learning-completed model may be distributed or executed through a recording medium, a network, or the like.

The learning-completed model has a data structure as described above. The learning-completed model is a model subjected to learning with learning data as described above. Note that, the learning-completed model may be configured to be able to subjected to further learning with further input of learning data.

### Other Embodiments

For example, a transmitter, a receiver, or an information processing device may be constituted by a plurality of devices. Specifically, processing and control may be performed in virtualization, parallelization, distribution, or redundancy. The devices of the transmitter, the receiver, and the information processing device may be integrated or shared as hardware.

The signal restoration system and the signal generation system may be configured to perform machine learning by using AI or the like. For example, each network structure may include a structure for performing machine learning, such as a generative adversarial network (GAN), a convolutional neural network (CNN), or a RNN.

Not both a configuration for the “learning processing” and a configuration for the “execution processing” may be included among functional configurations. For example, no configuration for the “execution processing” may be included at a stage where the “learning processing” is performed. Similarly, no configuration for the “learning processing” may be included at a stage where the “execution processing” is performed. A configuration different from that for processing to be performed may be excluded through such division into the stages of “learning” and “execution”. Note that, various settings of the network structure may be adjusted by a user, for example, after the “learning processing” or the “learning processing”.

Note that, all or some pieces of processing according to the present invention may be implemented by a computer program for causing a computer to execute a signal restoration method or a signal generation method, the computer program being described in a low-order language such as an assembler or a high-order language such as an object oriented language. In other words, the computer program is a computer program for causing a computer, such as the information processing device, the signal restoration system, or the signal generation system, to execute each processing.

Thus, when each processing is executed based on the computer program, an arithmetic device and a control device included in a computer perform calculation and control based on the computer program to execute the processing. In addition, a memory included in the computer stores data used for each processing based on the computer program to execute the processing.

The computer program may be recorded and distributed in a computer-readable recording medium. Note that, the recording medium is a medium such as a magnetic tape, a flash memory, an optical disk, a magneto optical disc, or a magnetic disk. The computer program may be distributed through an electric communication line.

Although preferable embodiments and the like are described above in detail, the present invention is not limited to the above-described embodiments and the like, and it is possible to subject the above-described embodiments and the like to modification and replacement in various kinds of manners without departing from a range written in the claims.

This application is based upon and claims the benefit of priority from Japanese Patent Application No. 2020-028681 filed on Feb. 21, 2020, the entire content of which is incorporated herein by reference.

### REFERENCE SIGNS LIST

1 signal restoration system

1F11 signal acquirer

1F12 first band-pass filter

1F13 integral calculator

1F14 second band-pass filter

1F15 first learning data generator

1F16 first learner

1F17 restored signal generator

1F21 fourth band-pass filter

1F22 second learning data generator

1F23 second learner

1F24 aortic pulse wave generator

1F25 blood pressure estimation unit

12 Doppler radar

12Rx receiver

12S source

12Tx transmitter

13 filter

IDX1 first indicator

IDX2 second indicator

IDX3 third indicator

L1 input

L2 multi-layer Bi-LSTM

L3 affine layer

L4 output

MDL learning model

P11 eleventh apex

P12 twelfth apex

P13 thirteenth apex

P14 fourteenth apex

P21 twenty-first apex

P22 twenty-second apex

P23 twenty-third apex

P24 twenty-four apex

PK1 first peak point

PK2 second peak point

PK3 third peak point

PWS aortic pulse wave signal

R1 comparative example

R2 proposed method

x displacement

θ phase

ω_{d} angular frequency

1. A signal restoration system comprising:
a signal acquirer configured to acquire a first heartbeat signal representing heartbeat behavior;
a first band-pass filter configured to generate a first signal by performing first band-pass filter processing on the first heartbeat signal;
an integral calculator configured to calculate an integral value by integrating frequency intensity of the heartbeat represented by the first signal;
a second band-pass filter configured to generate a third signal by performing second band-pass filter processing on a second signal representing the integral value with respect to time; and
a restored signal generator configured to generate a restored signal representing heartbeat behavior based on first data generated by dividing the third signal at intervals of a predetermined time.

2. The signal restoration system according to claim 1, wherein the restored signal generator generates the restored signal in which a Q wave, an R wave, an S wave, and a T wave in one period of heartbeat are restored or enhanced.

3. The signal restoration system according to claim 1, wherein the signal acquirer acquires the first heartbeat signal by a Doppler radar.

4. The signal restoration system according to claim 1, further comprising a spectrogram conversion unit configured to generate, based on the first signal, a spectrogram representing a relation between time and frequency intensity included in the first signal, wherein the integral calculator calculates the integral value by integrating the frequency intensity represented by the spectrogram.

5. The signal restoration system according to claim 1, wherein a frequency band excluded as an attenuation target is set to be wider for the first band-pass filter processing than for the second band-pass filter processing.

6. The signal restoration system according to claim 5, wherein
the first band-pass filter processing attenuates a frequency band other than 8 to 30 Hz, and
the second band-pass filter processing attenuates a frequency band other than 0.5 to 10.0 Hz.

7. The signal restoration system according to claim 1, wherein the restored signal generator includes an LSTM.

8. The signal restoration system according to claim 7, wherein the LSTM has a three-layer structure.

9. The signal restoration system according to claim 7, wherein the LSTM is a Bi-LSTM having a bidirectional configuration.

10. The signal restoration system according to claim 7, further comprising a parameter setting unit configured to set a parameter of the restored signal generator by machine learning using the LSTM.

11. A signal generation system comprising:
a signal acquirer configured to acquire a third heartbeat signal representing heartbeat behavior;
a fourth band-pass filter configured to generate a fourth signal by performing fourth band-pass filter processing on the third heartbeat signal;
an aortic pulse wave generator configured to generate, based on second data generated by dividing the fourth signal at intervals of a predetermined time, an aortic pulse wave signal including an aortic pulse wave or obtained by enhancing the aortic pulse wave; and
a blood pressure estimation unit configured to estimate blood pressure based on a parameter represented by the aortic pulse wave signal.

12. A signal restoration method executed by a signal restoration system, the signal restoration method comprising:
a signal acquisition procedure of acquiring, by a signal restoration system, a first heartbeat signal representing heartbeat behavior;
a first band-pass filter procedure of generating, by the signal restoration system, a first signal by performing first band-pass filter processing on the first heartbeat signal;
an integral calculation procedure of calculating, by the signal restoration system, an integral value by integrating frequency intensity of the heartbeat represented by the first signal;
a second band-pass filter procedure of generating, by the signal restoration system, a third signal by performing second band-pass filter processing on a second signal representing the integral value with respect to time; and
a restored signal generation procedure of generating, by the signal restoration system, a restored signal representing heartbeat behavior based on first data generated by dividing the third signal at intervals of a predetermined time.

13. A computer program for executing the signal restoration method according to claim 12.
